SEQUENCE LISTING (55 sequences)

SEQ ID NO: 1 - protein, 18 aa, synthetic construct
  VARIANT 4: note = X at this position for this sequence is referred to as "v" in the Specification for this sequence, and v is Q, D or E.
  VARIANT 11: note = X at this position for this sequence is referred to as "w" in the Specification for this sequence, and w is S or N.
  VARIANT 12: note = X at this position for this sequence is referred to as "x" in the Specification for this sequence, and x is H, N, or I.
  VARIANT 13: note = X at this position for this sequence is referred to as "y" in the Specification for this sequence, and y is D, A, I, N, G, H, K, S, or null.
  VARIANT 14: note = X at this position for this sequence is referred to as "z" in the Specification for this sequence, and z can be GGKQALETVQRLLPVLCQD or GGKQALETVQRLLPVLCQA.
LTPXQVVAIAXXXXGHGG

SEQ ID NO: 2 - protein, 18 aa, synthetic construct
  VARIANT 4: note = X at this position for this sequence is referred to as "v" in the Specification for this sequence, and v is Q, D or E.
  VARIANT 11: note = X at this position for this sequence is referred to as "w" in the Specification for this sequence, and w is S or N.
  VARIANT 12: note = X at this position for this sequence is referred to as "x" in the Specification for this sequence, and x is H, N, or I.
  VARIANT 13: note = X at this position for this sequence is referred to as "y" in the Specification for this sequence, and y is D, A, I, N, G, H, K, S, or null.
  VARIANT 14: note = X at this position for this sequence is referred to as "z" in the Specification for this sequence, and z can be GGKQALETVQRLLPVLCQD or GGKQALETVQRLLPVLCQA.
LTPXQVVAIAXXXXGTHG

SEQ ID NO: 3 - protein, 19 aa, synthetic construct
GGKQALETVQRLLPVLCQD

SEQ ID NO: 4 - protein, 19 aa, synthetic construct
GGKQALETVQRLLPVLCQA

SEQ ID NO: 5 - protein, 15 aa, synthetic construct
  VARIANT 4: note = X at this position for this sequence is referred to as "v" in the Specification for this sequence, and v is Q, D or E.
  VARIANT 11: note = X at this position for this sequence is referred to as "w" in the Specification for this sequence, and w is S or N.
  VARIANT 12: note = X at this position for this sequence is referred to as "x" in the Specification for this sequence, and x is H, N, or I.
  VARIANT 13: note = X at this position for this sequence is referred to as "y" in the Specification for this sequence, and y is D, A, I, N, G, H, K, S, or null.
  VARIANT 14: note = X at this position for this sequence is referred to as "z" in the Specification for this sequence, and z can be GGRPALE, GGKQALE, GKQALETVQRLLPVLCQDHG, GGKQALETVQRLLPVLCQAHG, GKQALETVQRLLPVLCQDHG, GKQALETVQRLLPVLCQAHG, GGKQALETVQRLLPVLCQD, or GGKQALETVQRLLPVLCQA.
  VARIANT 15: note = X at this position in the sequence is referred to as "alpha" in the Specification for this sequence, and alpha is four consecutive amino acids.
LTPXQVVAIAXXXXX

SEQ ID NO: 6 - protein, 7 aa, synthetic construct
GGRPALE

SEQ ID NO: 7 - protein, 7 aa, synthetic construct
GGKQALE

SEQ ID NO: 8 - protein, 20 aa, synthetic construct
GKQALETVQRLLPVLCQDHG

SEQ ID NO: 9 - protein, 21 aa, synthetic construct
GGKQALETVQRLLPVLCQAHG

SEQ ID NO: 10 - protein, 20 aa, synthetic construct
GKQALETVQRLLPVLCQDHG

SEQ ID NO: 11 - protein, 20 aa, synthetic construct
GKQALETVQRLLPVLCQAHG

SEQ ID NO: 12 - protein, 19 aa, synthetic construct
GGKQALETVQRLLPVLCQD

SEQ ID NO: 13 - protein, 19 aa, synthetic construct
GGKQALETVQRLLPVLCQA

SEQ ID NO: 14 - protein, 4 aa, synthetic construct
GHGG

SEQ ID NO: 15 - protein, 4 aa, synthetic construct
HGSG

SEQ ID NO: 16 - protein, 4 aa, synthetic construct
HGGG

SEQ ID NO: 17 - protein, 4 aa, synthetic construct
GGHD

SEQ ID NO: 18 - protein, 4 aa, synthetic construct
GAHD

SEQ ID NO: 19 - protein, 4 aa, synthetic construct
AHDG

SEQ ID NO: 20 - protein, 4 aa, synthetic construct
PHDG

SEQ ID NO: 21 - protein, 4 aa, synthetic construct
GPHD

SEQ ID NO: 22 - protein, 4 aa, synthetic construct
GHGP

SEQ ID NO: 23 - protein, 4 aa, synthetic construct
PHGG

SEQ ID NO: 24 - protein, 4 aa, synthetic construct
PHGP

SEQ ID NO: 25 - protein, 4 aa, synthetic construct
AHGA

SEQ ID NO: 26 - protein, 4 aa, synthetic construct
LHGA

SEQ ID NO: 27 - protein, 4 aa, synthetic construct
VHGA

SEQ ID NO: 28 - protein, 4 aa, synthetic construct
IVHG

SEQ ID NO: 29 - protein, 4 aa, synthetic construct
IHGM

SEQ ID NO: 30 - protein, 4 aa, synthetic construct
RHGD

SEQ ID NO: 31 - protein, 4 aa, synthetic construct
RDHG

SEQ ID NO: 32 - protein, 4 aa, synthetic construct
RHGE

SEQ ID NO: 33 - protein, 4 aa, synthetic construct
HRGE

SEQ ID NO: 34 - protein, 4 aa, synthetic construct
RHGD

SEQ ID NO: 35 - protein, 4 aa, synthetic construct
HRGD

SEQ ID NO: 36 - protein, 4 aa, synthetic construct
GPYE

SEQ ID NO: 37 - protein, 4 aa, synthetic construct
NHGG

SEQ ID NO: 38 - protein, 4 aa, synthetic construct
THGG

SEQ ID NO: 39 - protein, 4 aa, synthetic construct
GTHG

SEQ ID NO: 40 - protein, 4 aa, synthetic construct
GSGS

SEQ ID NO: 41 - protein, 4 aa, synthetic construct
GSGG

SEQ ID NO: 42 - protein, 4 aa, synthetic construct
GGGG

SEQ ID NO: 43 - protein, 4 aa, synthetic construct
GRGG

SEQ ID NO: 44 - protein, 4 aa, synthetic construct
GKGG

SEQ ID NO: 45 - DNA, 829 bp, synthetic construct
  source 1..829: mol_type = other DNA
aagctttaatacgactcactatagggtttaaaaaagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtactcagatctcgagctcaagtagataaataaaaacccaaaccctgacgggtttaaaagaagagcgaattc

SEQ ID NO: 46 - DNA, 869 bp, synthetic construct
  source 1..869: mol_type = other DNA
aagctttaatacgactcactatagggtctaaaaaagccaccatggtgagcaagaaaaaagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtactcagatctcgagctcaagtagcaagtactcagatctcgagctcaagtagataaataaagaccccccagatgactctgggaattc

SEQ ID NO: 47 - DNA, 825 bp, synthetic construct
  source 1..825: mol_type = other DNA
aagctttaatacgactcactatagggccgagcgaagcgtaaaaaagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtactcagatctcgagctcaagtagacgcttcgctcaaattaaaagaagagcgaattc

SEQ ID NO: 48 - DNA, 1046 bp, synthetic construct
  source 1..1046: mol_type = other DNA
aagctttaatacgactcactatagggccgagcgaagcgtaaagaaaagaaaaagaaaaaagaaagcaaaaatgtgatcttgcttgtaaatacaattttgagaggttaataaattacaagtagtgctatttttgtatttaggttagctatttagctttacgttccaggatgcctagtggcagccccacaatatccaggaagccctctctgcggtttttcagattaggtagtcgaaaaacctaagaaatttaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtactcagatctcgagctcaagtagaaagaaaagaaaaagaaaaaagacgcttcgctcaaattaaaagaagagcgaattc

SEQ ID NO: 49 - DNA, 1446 bp, synthetic construct
  source 1..1446: mol_type = other DNA
aagctttaatacgactcactatagggccgagcgaagcgtaaagaaaagaaaaagaaaaaagcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataatatggccacaaccatgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtactcagatctcgagctcaagtagaaagaaaagaaaaagaaaaaagacgcttcgctcaaattaaaagaagagcgaattc

SEQ ID NO: 50 - DNA, 1560 bp, synthetic construct
  source 1..1560: mol_type = other DNA
aagctttaatacgactcactatagggccgagcgaagcgtaaaaaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtactcagatctcgagctcaagtagacgcttcgctcaaattaaaagaagagcgaattc

SEQ ID NO: 51 - DNA, 1598 bp, synthetic construct
  source 1..1598: mol_type = other DNA
aagctttaatacgactcactatagggccgagcgaagcgtaaagaaaagaaaaagaaaaaagttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtactcagatctcgagctcaagtagaaagaaaagaaaaagaaaaaagacgcttcgctcaaattaaaagaagagcgaattc

SEQ ID NO: 52 - DNA, 1598 bp, synthetic construct
  source 1..1598: mol_type = other DNA
aagctttaatacgactcactatagggccgagcgaagcgtaaagaaaagaaaaagaaaaaagttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtatcaaaaggagaagaactattcacaggagtagtaccaatattagtagaactagacggggacgtaaacggacataaattctcagtatcaggagaaggagaaggggacgcaacatacggaaaactaacattaaaatttatctgcacaacaggcaaactaccagtaccatggccaacgttggtaacaacactaacatacggagtgcaatgcttctcaagatacccagaccacatgaaacaacacgacttcttcaaatcagcaatgccagaaggatacgtacaagaaagaacaatattcttcaaagacgacggaaactacaaaacaagagcagaagtaaaatttgaaggagacacattagtaaacagaatagaattaaaaggaatagacttcaaagaggacggcaacatactaggacacaaactagaatacaactacaatagccacaacgtatacataatggcagacaaacaaaaaaatggaatcaaagtaaacttcaaaataagacacaacatagaggacggctcagtacaactagccgaccactaccaacaaaacacaccaataggggacggacctgtattactacccgacaaccactatctatcaacacaatcagcactatccaaagacccaaacgaaaaaagagatcacatggtactactagagttcgtaacagcagcaggaataacattaggaatggacgaactatacaaatactcagacctagaattaaaataaaaagaaaagaaaaagaaaaaagacgcttcgctcaaattaaaagaagagcgaattc

SEQ ID NO: 53 - DNA, 51 bp, synthetic construct
  source 1..51: mol_type = other DNA
gggtttnnnataaataaaaacccaaaccctgacgggtttaaaagaagagcg

SEQ ID NO: 54 - DNA, 39 bp, synthetic construct
  source 1..39: mol_type = other DNA
gggtctnnnataaataaagaccccccagatgactctggg

SEQ ID NO: 55 - DNA, 35 bp, synthetic construct
  source 1..35: mol_type = other DNA
gggccgagcgaagcgtnnnacgcttcgctcaaatt

(19) United States
(12) Patent Application Publication
(10) Pub. No.: US 2024/0035025 A1
(43) Pub. Date: Feb. 1, 2024

(54) CIRCULAR RNA

(71) Applicant: Factor Bioscience Inc., Cambridge, MA (US)

(72) Inventors: Matthew ANGEL, Cambridge, MA (US); Christopher ROHDE, Cambridge, MA (US); Aisha SVIHLA, Cambridge, MA (US)

(21) Appl. No.: 18/480,405

(22) Filed: Oct. 3, 2023

Related U.S. Application Data
(63) Continuation of application No. PCT/US2022/026564, filed on Apr. 27, 2022.
(60) Provisional application No. 63/180,387, filed on Apr. 27, 2021.

(51) Int. Cl. C12N 15/11; C07K 14/47; C12N 15/10; C12N 9/22

(52) U.S. Cl. CPC C12N 15/11; C07K 14/4702; C12N 15/102; C12N 9/22; C12N 2310/20

(57) ABSTRACT

The present disclosure relates, in part, to circular RNAs and engineered variants thereof.

### CROSS-REFERENCE TO RELATED APPLICATIONS

This application is a continuation of PCT Application No. PCT/US2022/026564, filed Apr. 27, 2022, which claims the benefit of US Provisional Patent Application. No. 63/180,387, filed Apr. 27, 2021. The entire contents of the aforementioned patent applications are incorporated herein by reference.

### INCORPORATION BY REFERENCE OF SEQUENCE LISTING

The present application is being filed along with a Sequence Listing in electronic format. The Sequence Listing is provided as a file entitled 61057-718.601.xml, created on or about Sep. 6, 2023, which is 66,455 bytes in size. The information in the electronic format of the Sequence Listing is incorporated by reference in its entirety.

### BACKGROUND

Linear RNA molecules have many applications in biological research and therapeutic development. However, the relatively short half-life of long linear RNA molecules can limit their use in applications where long-term protein translation is desired. Circular RNAs (circRNAs) have been reported to exist in various organisms, including C. elegans, Drosophila melanogaster, mice, and humans and have been explored for a applications including microRNA sponges and protein expression. Methods for synthesizing circRNAs include the use of a splint molecule to bring the 5′ and 3′ ends of linear RNA in close proximity for ligation and the use of ribozymatic methods in conjunction with self-splicing introns to covalently link the 5′ and 3′ ends of an in vitro-transcribed (iVT) RNA molecule. However, these methods suffer from low efficiency or result in cytotoxic byproducts that must be removed using high-performance liquid chromatography (HPLC). What is needed is improved circRNA compositions, and methods of making circRNAs.

### SUMMARY

Accordingly, the present disclosure relates to, in aspects, RNA molecules which are engineered to promote formation of Circular RNAs (circRNAs), methods for making circRNAs, and the circRNAs themselves.

In aspects, the present disclosure provides methods of making circRNA and the circRNA that result therefrom.

In aspects, the present disclosure provides methods of making circRNA that are substantially free of cytotoxic byproducts. In embodiments, the present disclosure provides methods of making circRNA that do not require a linear oligonucleotide (splint) to pre-orient the two reacting ends of a linear RNA to assist in ligation to yield a circRNA. In embodiments, the present disclosure provides methods of making circRNA that do not require ribozymes to yield a circRNA. In embodiments, the present disclosure provides methods of making circRNA that do not require HPLC-based purification, e.g. post-ligation.

An aspect of the present disclosure is a composition comprising a nucleic acid of the structure: 5′-X—Y-A-IRES-B—CDS—C—Y′—Z-3′. In this aspect, Y and Y′ each independently comprise one or more nucleotides and Y and Y′ are substantially complementary; X and Z each independently comprise one or more nucleotides and X and Z are not substantially complementary; IRES comprises an internal ribosome entry site; CDS comprises a coding sequence; and A, B, and C are each independently a spacer comprising one or more nucleotides or null.

In embodiments, the protein of interest is selected from Table 1, Table 2, or Table 3, inclusive of the protein product of any gene of Table 1, Table 2, or Table 3.

In some embodiments, the protein of interest is one or more gene-editing proteins comprising (i) a DNA-binding domain comprising a plurality of repeat sequences and at least one of the repeat sequences comprises the amino acid sequence: LTPvQVVAIAwxyzGHGG or LTPvQVVAIAwxyzGTHG and is between 36 and 39 amino acids long, wherein: “v” is Q, D or E, “w” is S or N, “x” is H, N, or I, “y” is D, A, I, N, G, H, K, S, or null, and “z” is GGKQALETVQRLLPVLCQD or GGKQALETVQRLLPVLCQA and (ii) a nuclease domain comprising a catalytic domain of a nuclease.

In various embodiments, the protein of interest is one or more gene-editing proteins comprising (i) a DNA-binding domain comprising a plurality of repeat sequences and at least one of the repeat sequences comprises the amino acid sequence: LTPvQVVAIAwxyzα and is between 36 and 39 amino acids long, wherein: v is Q, D or E, w is S or N, x is I, H, N, or I, y is D, A, I, N, H, K, S, G or null, z is GGRPALE, GGKQALE, GKQALETVQRLLPVLCQDHG, GGKQALETVQRLLPVLCQAHG, GKQALETVQRLLPVLCQDHG, GKQALETVQRLLPVLCQAHG, GGKQALETVQRLLPVLCQD or GGKQALETVQRLLPVLCQA, a is four consecutive amino acids; and (ii) a nuclease domain comprising a catalytic domain of a nuclease. In some cases, α is selected from GHGG, HGSG, HGGG, GGHD, GAHD, AHDG, PHDG, GPHD, GHGP, PHGG, PHGP, AHGA, LHGA, VHGA, IVHG, IHGM, RHGD, RDHG, RHGE, HRGE, RHGD, HRGD, GPYE, NHGG, THGG, GTHG, GSGS, GSGG, GGGG, Another aspect of the present disclosure is a pharmaceutical composition comprising any herein-disclosed nucleic acid composition or comprising the any herein-disclosed circRNA composition, and a pharmaceutically acceptable carrier, vehicle or excipient.

Yes another aspect of the present disclosure is a host cell comprising any herein-disclosed composition.

In an aspect, the present disclosure provides a method of making a circRNA. The method comprising a step of contacting any herein-disclosed nucleic acid composition with one or more RNA ligases to result in circularization of the nucleic acid and formation of the circRNA.

In another aspect, the present disclosure provides a method of expressing a protein of interest in a cell. The method comprising a step of contacting the cell with any herein-disclosed nucleic acid or any herein-disclosed circRNA composition.

In yet another aspect, the present disclosure provides a method of gene editing a target nucleic acid in a cell. The method comprising a step of contacting the cell with any herein-disclosed composition or any herein-disclosed pharmaceutical composition. In this aspect, the composition comprises a circRNA in which the CDS encodes one or more proteins of interest, the proteins of interest being one or more gene-editing proteins, optionally selected from a nuclease, a transcription activator-like effector nuclease (TALEN), a zinc-finger nuclease, a meganuclease, a nickase, a clustered regularly interspaced short palindromic repeat (CRISPR)-associated protein, CRISPR/Cas9, Cas9, xCas9, Cas12a (Cpf1), Cas13a, Cas14, CasX, CasY, a Class 1 Cas protein, a Class 2 Cas protein, MAD7, and a gene-editing protein comprising a repeat sequence comprising LTPvQVVAIAwxyzα, or a natural or engineered variant, family member, orthologue, fragment or fusion construct thereof. In this aspect, the gene-editing protein is directed to the target nucleic acid.

In embodiments, the target nucleic acid is a gene selected from Table 2 or encodes a peptide or protein selected from Table 1 or Table 3.

In some embodiments, the method further comprises reprogramming the cell, e.g., comprising contacting the cell with a herein-disclosed composition comprising a circRNA comprising a CDS encoding a reprogramming factor.

An aspect of the present disclosure is a method of reprogramming a cell. The method comprising a step of contacting the cell with any herein-disclosed composition or any herein-disclosed pharmaceutical composition. In this aspect, the composition comprises a circRNA and the CDS encodes one or more proteins of interest, the protein of interest being one or more reprogramming factors, optionally selected from Oct4, Sox2, Klf4, c-Myc, l-Myc, Tert, Nanog, Lin28, Glis1, Utf1, Aicda, miR200 micro-RNA, miR291 micro-RNA, miR294 micro-RNA and miR295 micro-RNA, miR302 micro-RNA, miR367 micro-RNA, miR369 micro-RNA, or a natural or engineered variant, family member, orthologue, fragment or fusion construct thereof. In some cases, the CDS encodes two, three, four, five, six, seven, eight, nine, ten, eleven, or more reprogramming factor(s).

In various embodiments, the method comprises (a) providing a differentiated or a non-pluripotent cell; (b) culturing the differentiated or a non-pluripotent cell; (c) transfecting the differentiated or a non-pluripotent cell with the circRNA.

In embodiments, the method further comprises gene-editing the cell, e.g., comprising contacting the cell with any herein composition comprising a circRNA in which the CDS encodes one or more proteins of interest, the proteins of interest being one or more gene-editing proteins, optionally selected from a nuclease, a transcription activator-like effector nuclease (TALEN), a zinc-finger nuclease, a meganuclease, a nickase, a clustered regularly interspaced short palindromic repeat (CRISPR)-associated protein, CRISPR/Cas9, Cas9, xCas9, Cas12a (Cpf1), Cas13a, Cas14, CasX, CasY, a Class 1 Cas protein, a Class 2 Cas protein, MAD7, and a gene-editing protein comprising a repeat sequence comprising LTPvQVVAIAwxyzα, or a natural or engineered variant, family member, orthologue, fragment or fusion construct thereof.

In embodiments, the gene-editing targets a nucleic acid which is a gene selected from Table 2 or which encodes a peptide or protein selected from Table 1 or Table 3.

Another aspect of the present disclosure is a method of treating a disease, disorder, or condition. The method comprising steps of (1) contacting a cell with any herein-disclosed composition or any herein-disclosed pharmaceutical composition and administering the cell to a patient in need thereof or (2) administering any herein-disclosed composition or any herein-disclosed pharmaceutical composition of to a patient in need thereof. In embodiments, the disease, disorder, or condition is selected from Table 1 or Table 3.

Any aspect or embodiment described herein can be combined with any other aspect or embodiment as disclosed herein.

### BRIEF DESCRIPTION OF THE DRAWINGS

FIG. 1 is a non-limiting illustration of enhancing RNA circularization via non-complementary and complementary sequence elements.

FIG. 2 shows the predicted secondary structure from various 5′ and 3′ RNA base additions. Circled region shows 5′ most nucleotide.

FIG. 3 is a gel showing the generation of circRNA (refer to FIG. 2 for conditions).

FIG. 4 shows RNA products during circRNA synthesis. Lane 1: in vitro transcription reaction. Lane 2: Overnight ligation of ivT product using T4 RNA Ligase 1. Lane 3: Ligation product following calf intestinal phosphatase treatment. Lane 4: Ligation product following calf intestinal phosphatase treatment and RNase R digestion of the linear products.

FIG. 5 shows transfection of primary human dermal fibroblasts were electroporated with circRNA. Images taken 24 h following electroporation are shown.

FIG. 6 shows transfection of primary human dermal fibroblasts were electroporated with circRNA. Images taken 48 h following electroporation are shown.

FIG. 7 shows transfection of primary human dermal fibroblasts were electroporated with circRNA. Images taken 72 h following electroporation are shown.

FIG. 8 shows mean cell fluorescence intensity vs. time. 120 k primary human dermal fibroblasts were electroporated with 750 ng of circular RNA with the indicated IRES or linear RNA encoding GFP. Cells were imaged at the indicated time point following electroporation and the mean intensity of each green cell was calculated. For clarity, at time point 72, the curves are, top to bottom: CVB3_v4; CVB3_v3, linear, EMCV, minE, and CrPV.

### DETAILED DESCRIPTION

The present disclosure is based, in part, on the discovery of methods to yield circRNA in an efficient manner.

In aspects, the present disclosure provides RNA that is engineered to yield circRNA, e.g. via non-complementary and complementary sequence elements.

In aspects, the present disclosure provides methods of making circRNA and the circRNA that result therefrom.

circRNAs and Compositions Comprising circRNAs

circRNAs comprise single-stranded RNAs that are joined head to tail. circRNAs were initially discovered in pathogenic genomes such as hepatitis D virus (HDV) and plant viroids. circRNAs may function as potential molecular markers of disease and may play an important role in the initiation and progression of human diseases, including in tumors.

CircRNAs of the present disclosure may be about 100 nucleotides in length, about 200 nucleotides in length, about 300 nucleotides in length, about 400 nucleotides in length, about 500 nucleotides in length, about 600 nucleotides in length, about 700 nucleotides in length, about 800 nucleotides in length, about 900 nucleotides in length, about 1000 nucleotides in length, about 1100 nucleotides in length, about 1200 nucleotides in length, about 1300 nucleotides in length, about 1400 nucleotides in length, about 1500 nucleotides in length, about 2000 nucleotides in length, about 2500 nucleotides in length, about 3000 nucleotides in length, about 3500 nucleotides in length, about 4000 nucleotides in length, about 4500 nucleotides in length, about 5000 nucleotides in length, about 5500 nucleotides in length, about 6000 nucleotides in length, about 6500 nucleotides in length, about 7000 nucleotides in length, about 7500 nucleotides in length, about 8000 nucleotides in length, about 8500 nucleotides in length, about 9000 nucleotides in length, about 9500 nucleotides in length, or about 10000 nucleotides in length. CircRNAs of the present disclosure may be at least about 100 nucleotides in length, at least about 200 nucleotides in length, at least about 300 nucleotides in length, at least about 400 nucleotides in length, at least about 500 nucleotides in length, at least about 600 nucleotides in length, at least about 700 nucleotides in length, at least about 800 nucleotides in length, at least about 900 nucleotides in length, at least about 1000 nucleotides in length, at least about 1100 nucleotides in length, at least about 1200 nucleotides in length, at least about 1300 nucleotides in length, at least about 1400 nucleotides in length, at least about 1500 nucleotides in length, at least about 1600 nucleotides in length, at least about 1700 nucleotides in length, at least about 1800 nucleotides in length, at least about 1900 nucleotides in length, at least about 2000 nucleotides in length, at least about 2500 nucleotides in length, at least about 3000 nucleotides in length, at least about 3500 nucleotides in length, at least about 4000 nucleotides in length, at least about 4500 nucleotides in length, at least about 5000 nucleotides in length, at least about 5500 nucleotides in length, at least about 6000 nucleotides in length, at least about 6500 nucleotides in length, at least about 7000 nucleotides in length, at least about 7500 nucleotides in length, at least about 8000 nucleotides in length, at least about 8500 nucleotides in length, at least about 9000 nucleotides in length, at least about 9500 nucleotides in length, or at least about 10000 nucleotides in length. CircRNAs of the present disclosure may be from about 100 to about 200 nucleotides in length, from about 200 to about 300 nucleotides in length, from about 300 to about 400 nucleotides in length, from about 400 to about 500 nucleotides in length, from about 500 to about 600 nucleotides in length, from about 600 to about 700 nucleotides in length, from about 700 to about 800 nucleotides in length, from about 800 to about 900 nucleotides in length, from about 900 to about 1000 nucleotides in length, from about 1000 to about 1100 nucleotides in length, from about 1100 to about 1200 nucleotides in length, from about 1200 to about 1300 nucleotides in length, from about 1300 to about 1400 nucleotides in length, from about 1400 to about 1500 nucleotides in length, from about 1500 to about 2000 nucleotides in length, from about 2000 to about 2500 nucleotides in length, from about 2500 to about 3000 nucleotides in length, from about 3000 to about 3500 nucleotides in length, from about 3500 to about 4000 nucleotides in length, from about 4000 to about 4500 nucleotides in length, from about 4500 to about 5000 nucleotides in length, from about 5000 to about 5500 nucleotides in length, from about 5500 to about 6000 nucleotides in length, from about 6000 to about 6500 nucleotides in length, from about 6500 to about 7000 nucleotides in length, from about 7000 to about 7500 nucleotides in length, from about 7500 to about 8000 nucleotides in length, from about 8000 to about 8500 nucleotides in length, from about 8500 to about 9000 nucleotides in length, from about 9000 to about 9500 nucleotides in length, or from about 9500 to about 10000 nucleotides in length, and any length therebetween.

Preparation of circRNAs from linear precursors can pose serious challenges, due to, inter alia, a negative entropy associated with the circularization step. The most significant side reaction can be intramolecular bond forming leading to oligomerization of the linear precursor instead of circularization.

In aspects, the present disclosure relates to engineered nucleic acids, e.g. RNAs, that are suitable for efficient circularization to yield circRNAs. In aspects, the present disclosure relates to RNA sequences that form a hairpin loop in close proximity to the 5′ and 3′ ends and better support intramolecular ligation than RNA sequences without such a hairpin loop.

In aspects, the present disclosure relates to a composition comprising a nucleic acid of the structure:

5′-X—Y-A-IRES-B—CDS—C—Y′—Z-3′,

where Y and Y′ each independently comprise one or more nucleotides and Y and Y′ are substantially complementary; X and Z each independently comprise one or more nucleotides and X and Z are not substantially complementary; IRES comprises an internal ribosome entry site; CDS comprises a coding sequence; and A, B, and C are each independently a spacer comprising one or more nucleotides or null.

In embodiments, the nucleic acid is RNA. In embodiments, the RNA is synthetic RNA.

In embodiments, the nucleic acid forms a circular RNA (circRNA), e.g., when contacted with an RNA ligase.

In embodiments, the CDS comprises one more exons. In embodiments, the CDS encodes one or more proteins of interest. In embodiments, the protein of interest is a soluble protein. In embodiments, the protein of interest is selected from Table 1, Table 2, or Table 3, inclusive of the protein product of any gene of Table 1, Table 2, or Table 3.

In embodiments, the protein of interest is one or more reprogramming factors, optionally selected from Oct4, Sox2, Klf4, c-Myc, l-Myc, Tert, Nanog, Lin28, Glis1, Utf1, Aicda, miR200 micro-RNA, miR291 micro-RNA, miR294 micro-RNA and miR295 micro-RNA, miR302 micro-RNA, miR367 micro-RNA, miR369 micro-RNA, or a natural or engineered variant, family-member, orthologue, fragment or fusion construct thereof.

In embodiments, the protein of interest is one or more gene-editing proteins, optionally selected from a nuclease, a transcription activator-like effector nuclease (TALEN), a zinc-finger nuclease, a meganuclease, a nickase, a clustered regularly interspaced short palindromic repeat (CRISPR)-associated protein, CRISPR/Cas9, Cas9, xCas9, Cas12a (Cpf1), Cas13a, Cas14, CasX, CasY, a Class 1 Cas protein, a Class 2 Cas protein, and MAD7, or a natural or engineered variant, family member, orthologue, fragment or fusion construct thereof.

In embodiments, the protein of interest is one or more gene-editing proteins comprising (i) a DNA-binding domain comprising a plurality of repeat sequences and at least one of the repeat sequences comprises the amino acid sequence: LTPvQVVAIAwxyzGHGG or LTPvQVVAIAwxyzGTHG and is between 36 and 39 amino acids long, wherein: “v” is Q, D or E, “w” is S or N, “x” is H, N, or I, “y” is D, A, I, N, G, H, K, S, or null, and “z” is GGKQALETVQRLLPVLCQD or GGKQALETVQRLLPVLCQA and (ii) a nuclease domain comprising a catalytic domain of a nuclease.

In embodiments, the protein of interest is one or more gene-editing proteins comprising (i) a DNA-binding domain comprising a plurality of repeat sequences and at least one of the repeat sequences comprises the amino acid sequence: LTPvQVVAIAwxyzα and is between 36 and 39 amino acids long, wherein: v is Q, D or E, w is S or N, x is I, H, N, or I, y is D, A, I, N, H, K, S, G or null, z is GGRPALE, GGKQALE, GGKQALETVQRLLPVLCQDHG, GGKQALETVQRLLPVLCQAHG, GKQALETVQRLLPVLCQDHG, GKQALETVQRLLPVLCQAHG, GGKQALETVQRLLPVLCQD or GGKQALETVQRLLPVLCQA, a is four consecutive amino acids; and (ii) a nuclease domain comprising a catalytic domain of a nuclease. In embodiments, a is selected from

GHGG,
HGSG,
HGGG,
GGHD,
GAHD,
AHDG,
PHDG,
GPHD,
GHGP,
PHGG,
PHGP,
AHGA,
LHGA,
VHGA,
IVHG,
IHGM,
RHGD,
RDHG,
RHGE,
HRGE,
RHGD,
HRGD,
GPYE,
NHGG,
THGG,
GTHG,
GSGS,
GSGG,
GGGG,
GRGG,
and
GKGG.

In embodiments, the IRES is selected from cMyc, CVB3 (coxsackievirus B3), EMCV (encephalomyocarditis virus), HCV (hepatitis C virus) HRV (human rhinovirus), or a natural or engineered variant, family member, orthologue, fragment or fusion construct thereof. In embodiments, the IRES is selected from poliovirus (PV), encephalomyelocarditis virus (EMCV), classical swine-fever virus (CSFV), foot-and-mouth disease virus (FMDV), human immunodeficiency virus (HIV), bovine viral diarrhoea virus (BVDV) and cricket paralysis virus (CrPV), or a natural or engineered variant, family member, orthologue, fragment or fusion construct thereof.

In embodiments, the nucleic acid further comprises a Type IIS restriction enzyme site at or near the 3′ end.

In embodiments, A comprises one or more nucleotides, e.g. about 5-25, or about 5-20, or about 5-15, or about 5-15 nucleotides, e.g. about 5, or about 10, or about 15, or about 20, or about 25 nucleotides. In embodiments, A is null.

In embodiments, B comprises one or more nucleotides, e.g. about 5-25, or about 5-20, or about 5-15, or about 5-15 nucleotides, e.g. about 5, or about 10, or about 15, or about 20, or about 25 nucleotides. In embodiments, B is null.

In embodiments, C comprises one or more nucleotides, e.g. about 5-25, or about 5-20, or about 5-15, or about 5-15 nucleotides, e.g. about 5, or about 10, or about 15, or about 20, or about 25 nucleotides. In embodiments, C is null.

In embodiments, A comprises one or more nucleotides, e.g. about 1-200, or about 50-200, or about 100-200, or about 150-200 nucleotides, e.g. about 50, or about 75, or about 100, or about 150, or about 200 nucleotides.

In embodiments, B comprises one or more nucleotides, e.g. about 1-200, or about 50-200, or about 100-200, or about 150-200 nucleotides, e.g. about 50, or about 75, or about 100, or about 150, or about 200 nucleotides.

In embodiments, C comprises one or more nucleotides, e.g. about 1-200, or about 50-200, or about 100-200, or about 150-200 nucleotides, e.g. about 50, or about 75, or about 100, or about 150, or about 200 nucleotides.

In embodiments, A, B, and C are identical. In embodiments, two of A, B, and C are identical.

In embodiments, A, B, and C are non-identical.

In embodiments, Y and Y′ each independently comprise one or more nucleotides, e.g. about 1-25, or about 1-20, or about 1-15, or about 1-15 nucleotides, or about 5-10 nucleotides e.g. about 1, or about 2, or about 3, or about 4, or about 5, or about 10, or about 15, or about 20, or about 25 nucleotides.

In embodiments, X and Z each independently comprise one or more nucleotides, e.g. about 1-25, or about 1-20, or about 1-15, or about 1-15 nucleotides, or about 5-10 nucleotides e.g. about 1, or about 2, or about 3, or about 4, or about 5, or about 10, or about 15, or about 20, or about 25 nucleotides.

In embodiments, Y and Y′ are fully complementary. In embodiments, Y and Y′ are partially complementary.

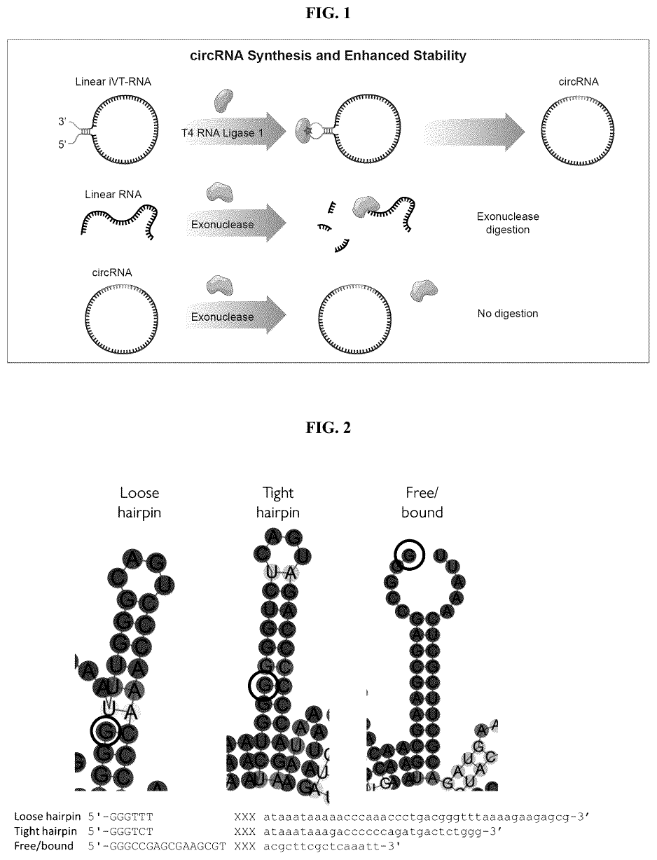

In embodiments, X, Z, Y and Y′ form a hairpin structure. In embodiments, the hairpin structure is a loose hairpin of 24 or fewer nucleotides being preferentially bound within the hairpin. In embodiments, the hairpin structure is a tight hairpin with more than 24 nucleotides being preferentially bound within the hairpin. In embodiments, X, Z, Y and Y′ do not form a hairpin structure. In embodiments, X, Z, Y and Y′ form one of the structures illustrated in of FIG. 2.

In embodiments, X and Z are suitable for interaction with an RNA ligase. In embodiments, the RNA ligase is a single strand RNA ligase. In embodiments, the single strand RNA ligase is T4 RNA Ligase 1, or a natural or engineered variant, family member, orthologue, fragment or fusion construct thereof.

In embodiments, the circRNA is substantially stable from an exonuclease.

In embodiments, the nucleic acid is suitable for synthesis by in vitro transcription.

In embodiments, the circRNA molecule has better pharmacokinetic behavior, such as absorption, efficacy, bioavailability and/or half-life, than a linear RNA molecule comprising the same sequence, or comparable sequence, as the circRNA molecule.

In embodiments, the circRNA molecule has better stability than a linear RNA molecule comprising the same sequence, or comparable sequence, as the circRNA molecule. In embodiments, the circRNA molecule has better intracellular stability than a linear RNA molecule comprising the same sequence, or comparable sequence, as the circRNA molecule.

In embodiments, the circRNA molecule has a longer intracellular half-life than a linear RNA molecule comprising the same sequence, or comparable sequence, as the circRNA molecule.

In embodiments, the intracellular half-life of the circRNA molecule is at least about 3 hours longer, or at least about 6 hours longer, or at least about 12 hours longer than a linear RNA molecule comprising the same sequence, or comparable sequence, as the circRNA molecule

In embodiments, the intracellular half-life of the circRNA molecule is at least about 1.5 times as long, or at least about 3 times as long, or at least about 5 times as long, or at least about 10 times as long, or at least about 30 times as long, or at least about 100 times as long as a linear RNA molecule comprising the same sequence, or comparable sequence, as the circRNA molecule.

In embodiments, there is provided a pharmaceutical composition comprising a composition comprising a herein-disclosed nucleic acid molecule (e.g., comprising the linear nucleic acid of the structure: 5′-X—Y-A-IRES-B—CDS—C—Y′—Z-3′), and a pharmaceutically acceptable carrier, vehicle or excipient.

In embodiments, there is provided a pharmaceutical composition comprising a composition comprising a herein-disclosed circRNA molecule, and a pharmaceutically acceptable carrier, vehicle or excipient.

In embodiments, there is provided a host cell comprising a herein-disclosed nucleic acid molecule (e.g., comprising the linear nucleic acid of the structure: 5′-X—Y-A-IRES-B—CDS—C—Y′—Z-3′) or a composition comprising the herein-disclosed nucleic acid.

In embodiments, there is provided a host cell comprising a herein-disclosed circRNA molecule or a composition comprising the herein-disclosed circRNA molecule.

Methods of Making

In embodiments, the present disclosure provides methods of making circRNA that are substantially free of cytotoxic byproducts.

In embodiments, the present disclosure provides methods of making circRNA that do not require a linear oligonucleotide (splint) to pre-orient the two reacting ends of a linear RNA to assist in ligation to yield a circRNA. In embodiments, the present disclosure provides methods of making circRNA that do not require ribozymes to yield a circRNA. In embodiments, the present disclosure provides methods of making circRNA that do not require a linear oligonucleotide (splint) to pre-orient the two reacting ends of a linear RNA to assist in ligation to yield a circRNA and do not require ribozymes to yield a circRNA. In embodiments, the present disclosure provides methods of making circRNA that have better ligation efficiency than alternative methods, e.g., those involving splints or ribozymes.

In embodiments, the present disclosure provides methods of making circRNA that do not require HPLC-based purification, e.g. post-ligation.

In embodiments, the present disclosure provides nucleic acids (e.g., a linear RNA molecules of the structure: 5′-X—Y-A-IRES-B—CDS—C—Y′—Z-3′) that form a hairpin loop in close proximity to the 5′ and 3′ ends and support intramolecular ligation, such intramolecular ligation being improved, e.g. relative to RNA sequences without such a hairpin loop.

In embodiments, the present disclosure provides a method of making a circRNA comprising contacting a herein-disclosed composition comprising a nucleic acid (e.g., a linear RNA molecule of the structure: 5′-X—Y-A-IRES-B—CDS—C—Y′—Z-3′) with one or more RNA ligases to result in circularization of the nucleic acid. In embodiments, the RNA ligase is a single strand RNA ligase. In embodiments, the single strand RNA ligase is T4 RNA Ligase 1, or a natural or engineered variant, family member, orthologue, fragment or fusion construct thereof.

In embodiments, the nucleic acid (e.g., a linear RNA molecule of the structure: 5′-X—Y-A-IRES-B—CDS—C—Y′—Z-3′) is synthesized in vitro transcription (iVT). In embodiments, the nucleic acid is synthesized using both guanosine monophosphate and guanosine triphosphate in the iVT reaction. In embodiments guanosine monophosphate and guanosine triphosphate are present at a ratio e.g. about 1:1, or about 2:1, or about 5:1, or about 10:1 or about 20:1.

In embodiments, the nucleic acid and/or circRNA comprises at least one non-canonical nucleotide. In embodiments, the non-canonical nucleotide is selected from 5-methylcytidine, 5-hydroxycytidine, 5-hydroxymethylcytidine, 5-carboxycytidine, 5-formylcytidine, 5-methoxy cytidine, pseudouridine, 5-hydroxyuridine, 5-methyluridine, 5-hydroxymethyluridine, 5-carboxyuridine, 5-methoxyuridine, 5-formyluridine, 5-hydroxypseudouridine, 5-methylpseudouridine, 5-hydroxymethylpseudouridine, 5-carboxypseudouridine, 5-methoxypseudouridine, and 5-formylpseudouridine

In embodiments, the nucleic acid and/or circRNA lacks any non-canonical nucleotides.

Methods of Expressing a Protein of Interest/Reprogramming/Gene-Editing/Disease Treatment

In embodiments, the present disclosure provides a method of expressing a protein of interest in a cell, comprising contacting the cell with a herein-disclosed composition comprising a circRNA.

In embodiments, the cell is contacted with mild hypothermic conditions, e.g., about 30° C. to about 36° C.

In embodiments, the present disclosure provides a method of gene editing a target nucleic acid in a cell, comprising contacting the cell with the composition or pharmaceutical composition described herein where the composition comprises a circRNA and the CDS encodes one or more proteins of interest, the proteins of interest being one or more gene-editing proteins, optionally selected from a nuclease, a transcription activator-like effector nuclease (TALEN), a zinc-finger nuclease, a meganuclease, a nickase, a clustered regularly interspaced short palindromic repeat (CRISPR)-associated protein, CRISPR/Cas9, Cas9, xCas9, Cas12a (Cpf1), Cas13a, Cas14, CasX, CasY, a Class 1 Cas protein, a Class 2 Cas protein, MAD7, and a gene-editing protein comprising a repeat sequence comprising LTPvQVVAIAwxyzα as defined herein, or a natural or engineered variant, family member, orthologue, fragment or fusion construct thereof, where the gene-editing protein is directed to the target nucleic acid. In some cases, the target nucleic acid is a gene selected from Table 2 or encodes a peptide or protein selected from Table 1 or Table 3. In embodiments, the present disclosure provides a method of increasing a replicative potential of a cell, comprising contacting the cell with the composition or pharmaceutical composition described herein where the composition comprises a circRNA and the CDS encodes one or more proteins of interest. In embodiments, the circRNA encodes a protein that increases the replicative potential of a cell. In embodiments, the encoded protein is TERT. In embodiments, the cell is selected from: a hematopoietic cell, a hematopoietic stem cell, a T cell, an NK cell, a myeloid cell, a macrophage, a tumor-infiltrating lymphocyte, a marrow-infiltrating lymphocytes, a peripheral blood lymphocyte, and a hair follicle stem cell.

In embodiments, the circRNA encodes a protein that increases the interaction between a cell and a protein. In embodiments, the encoded protein is chimeric antigen receptor. In embodiments, the cell is selected from: a hematopoietic cell, a hematopoietic stem cell, a T cell, an NK cell, a myeloid cell, a macrophage, a tumor-infiltrating lymphocyte, a marrow-infiltrating lymphocytes, a peripheral blood lymphocyte, and a hair follicle stem cell.

In embodiments, the cell is contacted with mild hypothermic conditions, e.g., about 30° C. to about 36° C.

In embodiments, the method further comprises reprogramming the cell. In embodiments, the present disclosure provides a method of reprogramming a cell, comprising contacting the cell with the composition described herein, where the composition is a circRNA and the CDS encodes one or more proteins of interest, the protein of interest being one or more (e.g., two, three, four, five, six, seven, eight, nine, ten, eleven, or more) reprogramming factors, optionally selected from Oct4, Sox2, Klf4, c-Myc, l-Myc, Tert, Nanog, Lin28, Glis1, Utf1, Aicda, miR200 micro-RNA, miR291 micro-RNA, miR294 micro-RNA and miR295 micro-RNA, miR302 micro-RNA, miR367 micro-RNA, miR369 micro-RNA, or a natural or engineered variant, family member, orthologue, fragment or fusion construct thereof.

In embodiments, method comprises (a) providing a differentiated or a non-pluripotent cell; (b) culturing the differentiated or a non-pluripotent cell; (c) transfecting the differentiated or a non-pluripotent cell with the circRNA. In embodiments, the transfecting is accomplished via electroporation. In embodiments, step (c) occurs in the presence of a medium containing ingredients that support reprogramming of the differentiated or a non-pluripotent to a less differentiated state. In embodiments, the method further comprises repeating step (c) at least twice during 5 consecutive days. In embodiments, the amount of one or more circRNA molecules transfected in one or more later transfections is greater than the amount transfected in one or more earlier transfections. In embodiments, steps (a)-(c) are performed without using feeder cells and occur in the presence of a feeder cell conditioned medium. In embodiments, step (c) is performed without using irradiated human neonatal fibroblast feeder cells and occurs in the presence of a feeder cell conditioned medium. In embodiments, the circRNA molecule encodes one or more (e.g., two, three, four, five, six, seven, eight, nine, ten, eleven, or more) reprogramming factor(s) selected from Oct4, Sox2, Klf4, c-Myc, l-Myc, Tert, Nanog, Lin28, Glis1, Utf1, Aicda, miR200 micro-RNA, MiR291 micro-RNA, miR294 micro-RNA and miR295 micro-RNA, miR302 micro-RNA, miR367 micro-RNA, MiR369 micro-RNA and biologically active fragments, analogues, variants and family members thereof. In embodiments, the differentiated or a non-pluripotent cell is derived from a biopsy. In embodiments, the differentiated or a non-pluripotent cell is from a human subject. In embodiments, the differentiated or a non-pluripotent cell is derived from a dermal punch biopsy sample. In embodiments, the differentiated or a non-pluripotent cell is a skin cell (e.g., a fibroblast or a keratinocyte). In embodiments, the method comprises or further comprises contacting the cell with at least one member of the group: poly-L-lysine, poly-L-ornithine, RGD peptide, fibronectin, vitronectin, collagen, and laminin. In embodiments, the method further comprises gene-editing the cell.

In embodiments, the circRNA molecule comprises at least one non-canonical nucleotide, optionally selected from 5-methylcytidine, 5-hydroxycytidine, 5-hydroxymethylcytidine, 5-carboxycytidine, 5-formylcytidine, 5-methoxycytidine, pseudouridine, 5-hydroxyuridine, 5-methyluridine, 5-hydroxymethyluridine, 5-carboxyuridine, 5-methoxyuridine, 5-formyluridine, 5-hydroxypseudouridine, 5-methylpseudouridine, 5-hydroxymethylpseudouridine, 5-carboxypseudouridine, 5-methoxypseudouridine, and 5-formylpseudouridine.

In embodiments, the circRNA molecule lacks any non-canonical nucleotides.

In embodiments, the medium is substantially free of immunosuppressants.

In embodiments, the cell is contacted with mild hypothermic conditions, e.g., about 30° C. to about 36° C.

In embodiments, the method further comprises gene-editing the cell.

In embodiments, the present disclosure provides a method of treating a disease, disorder, or condition comprising: (a) contacting a cell with the composition described herein (e.g., comprising a circRNA) and administering the cell to a patient in need thereof or (b) administering the composition (e.g., comprising a circRNA) to a patient in need thereof. In embodiments, the disease, disorder, or condition is selected from Table 1 or Table 3.

In embodiments, the circRNA molecule encodes one or more of: a T-cell receptor, a chimeric antigen receptor, a bispecific T-cell engagers (BiTE), a checkpoint inhibitor, an antibody, a nanobody, or a natural or engineered variant, family member, orthologue, fragment or fusion construct thereof.

In some cases, a cell transfected with a circRNA is a T-cell and the circRNA encodes a chimeric antigen receptor such that the transfected cell becomes a CAR-T. In other cases, a cell transfected with a circRNA is an NK cell and the circRNA encodes a chimeric antigen receptor such that the transfected cell becomes a CAR-NK.

In various cases, a reprogrammed cell (with or without gene editing) is provided to a subject in need thereof in the context of a stem cell therapy.

In embodiments, the circRNA molecule encodes on or more of Interleukin 7, Interleukin 12, Interleukin 15, Interleukin 18, dominant-negative TGF-β receptor II (dnTGF-βRII), constitutively active Akt (caAkt), or CD40 Ligand (CD40L), or a natural or engineered variant, family member, orthologue, fragment or fusion construct thereof.

In embodiments, the disease/indication is associated with one or more cancers. The one or more cancers may comprise: adenoid cystic carcinoma, adrenal gland tumor, amyloidosis, anal cancer, appendix cancer, astrocytoma, ataxia-telangiectasia, Beckwith-Wiedemann Syndrome, bile duct caner (Cholangiocarcinoma), Birt-Hogg Dube Syndrome, bladder cancer, bone cancer (sarcoma of bone), brain stem glioma, brain tumor, breast cancer, breast cancer (inflammatory), breast cancer (metastatic), breast cancer in men, camey complex, central nervous system tumors (brain and spinal cord), cervical cancer, childhood cancer, colorectal cancer, Cowden Syndrome, craniopharyngioma, desmoid tumor, desmoplastic infantile ganglioglioma tumor, ependymoma, esophageal cancer, Ewing Sarcoma, eye cancer, eyelid cancer, familial adenomatous polyposis, familial GIST, familial malignant melanoma, familial pancreatic cancer, gallbladder cancer, gastrointestinal stromal tumor GIST, germ cell tumor, gestational trophoblastic disease, head and neck cancer, hereditary breast and ovarian cancer, hereditary diffuse gastric cancer, hereditary leiomyomatosis and renal cell cancer, hereditary mixed polyposis syndrome, hereditary pancreatitis, hereditary papillary renal carcinoma, HIV/AIDS related cancer, juvenile polyposis syndrome, kidney cancer, lacrimal gland tumor, laryngeal and hypopharyngeal cancer, leukemia—acute lymphoblastic—ALL, leukemia, acute lymphocytic—ALL, leukemia—acute myeloid—ALL, leukemia—acute myeloid—AML, leukemia—B-cell prolymphocytic leukemia and hairy cell leukemia, leukemia—chronic lymphocytic—CLL, leukemia—chronic myeloid—CML, leukemia—chronic t-cell lymphocytic, leukemia—eosinophilic, Li-Fraumeni Syndrome, liver cancer, lung cancer—non-small cell, lung cancer—small cell, lymphoma—Hodgkin, lymphoma—Non-Hodgkin, Lynch Syndrome, mastocytosis, medulloblastoma, melanoma, meningioma, mesothelioma, multiple endocrine neoplasia type 1, multiple endocrine neoplasia type 2, multiple myeloma, MUTYH/MYH—associated polyposis, myelodysplastic syndromes—MDS, nasal cavity and paranasal sinus cancer, nasopharyngeal cancer, neuroblastoma, neuroendocrine tumor of the gastrointestinal tract, neuroendocrine tumor of the lung, neuroendocrine tumor of the pancreas, neuroendocrine tumors, neurofibromatosis type 1, neurofibromatosis type 2, nevoid basal cell carcinoma syndrome, oral and oropharyngeal cancer, osteosarcoma, ovarian cancer, fallopian tube cancer, peritoneal cancer, pancreatic cancer, parathyroid cancer, penile cancer, Peutz-Jeghers Syndrome, pheochromocytoma and paraganglioma, pituitary gland tumor, pleuropulmonary blastoma, prostate cancer, retinoblastoma, rhabdomyosarcoma, salivary gland cancer, sarcoma—Kaposi, sarcomas of specific organs, sarcomas—soft tissue, skin cancer (non-melanoma), skin cancer (melanoma), small bowel cancer, stomach cancer, testicular cancer, thyoma and thymic carcinoma, thyroid cancer, tuberous sclerosis complex, unknown primary, uterine cancer, vaginal cancer, Von Hippel-Lindau Syndrome, vulvar cancer, Waldenstrom macroglobulinemia (lymphoplasmacytic lymphoma), Werner Syndrome, Wilms tumor, and xeroderma pigmentosum.

In embodiments, gene editing further comprises transfecting a cell with a nucleic acid that acts as a repair template by either causing the insertion of a DNA sequence in the region of a gene edit, e.g., a single-strand or double-strand break, or by causing the DNA sequence in the region of the gene edit to otherwise change.

In some cases, the gene edit targets a genomic safe harbor locus, e.g., TRAC and AAVS1.

In various embodiments, a circRNA encodes a protein of interest that serves as a vaccine when introduced into a subject in need of vaccination.

The circRNA and nucleic acids of the present disclosure may be adapted to and use in the methods or compositions described in WO/2013/086008, WO/2014/071219, WO/2015/117021, WO/2016/131052, WO/2018/035377, WO/2019/191341, WO/2021/003462, WO2021/231549, or WO2021/222389. The entire contents of which are incorporated by reference in their entirety.

TABLE 1
Illustrative Indications, Proteins, and Peptides

| Illustrative | |
|---|---|
| Indication | Illustrative Protein/Peptide |
| Acne | Retinol Dehydrogenase 10 |
| Aging | Elastin, sp\|P15502\|ELN_HUMAN Elastin, (isoform 3), |
| Aging | Collagen Type I, P02452\|CO1A1_HUMAN Collagen alpha-1(I) chain, |
| | (P08123\|CO1A2_HUMAN Collagen alpha-2(I) chain, |
| Aging | Collagen Type III, P02461\|CO3A1_HUMAN Collagen alpha-1(III) chain, |
| | (isoform 1), |
| Aging | Collagen Type VII, Q02388\|CO7A1_HUMAN Collagen alpha-1(VII) chain, |
| Aging | Hyaluronan Synthase |
| Aging | Telomerase Reverse Transcriptase |
| Aging | Oct4, Sox2, Klf4, c-Myc, 1-Myc, Tert, Nanog, Lin28, Glis1, Utf1, Aicda, |
| | miR200 micro-RNA, miR291 micro-RNA, miR294 micro-RNA and miR295 |
| | micro-RNA, miR302 micro-RNA, miR367 micro-RNA, or miR369 micro-RNA |
| Albinism | Tyrosinase, P14679\|TYRO_HUMAN Tyrosinase, (isoform 1), |
| Alport Syndrome | Collagen Type IV; P02462\|CO4A1_HUMAN Collagen alpha-1(IV) chain, |
| | (isoform 1), P08572\|CO4A2_HUMAN Collagen alpha-2(IV) chain, |
| | Q01955\|CO4A3_HUMAN Collagen alpha-3(IV) chain, (isoform 1), |
| | P53420\|CO4A4_HUMAN Collagen alpha-4(IV) chain, |
| | P29400\|CO4A5_HUMAN Collagen alpha-5(IV) chain, (isoform 1), |
| | Q14031\|CO4A6_HUMAN Collagen alpha-6(IV), (isoform A), |
| Anemia | Erythropoietin |
| Atopic Dermatitis | Filaggrin |
| Cancers (as | T-cell receptor, a chimeric antigen receptor, a bispecific T-cell engagers (BiTE), |
| disclosed herein) | a checkpoint inhibitor, an antibody, a nanobody, or a natural or engineered |
| | variant, family member, orthologue, fragment or fusion construct thereof, |
| | Interleukin 7, Interleukin 12, Interleukin 15, Interleukin 18, dominant-negative |
| | TGF-β receptor II (dnTGF-βRII), constitutively active Akt (caAkt), or CD40 |
| | Ligand (CD40L) |
| Cutis Laxa | Elastin, sp\|P15502\|ELN_HUMAN Elastin, (isoform 3), |
| Dry Skin | Filaggrin |
| Dystrophic | Collagen Type VII; Q02388\|CO7A1_HUMAN Collagen alpha-1(VII) chain, |
| Epidermolysis | |
| Bullosa | |
| Ehlers-Danlos | Collagen Type V; P20908\|CO5A1_HUMAN Collagen alpha-1(V) chain, |
| Syndrome | P05997\|CO5A2_HUMAN Collagen alpha-2(V) chain, |
| | P25940\|CO5A3_HUMAN Collagen alpha-3(V) chain, |
| Ehlers-Danlos | Collagen Type I, P02452\|CO1A1_HUMAN Collagen alpha-1(I) chain, |
| Syndrome | P08123\|CO1A2_HUMAN Collagen alpha-2(I) chain, |
| Epidermolysis | ADAM17, P78536\|ADA17_HUMAN Disintegrin and metalloproteinase |
| bullosa, lethal | domain-containing protein 17, (isoform A), |
| acantholytic | |
| Epidermolysis | Collagen Type III, P02461\|CO3A1_HUMAN Collagen alpha-1(III) chain, |
| bullosa, type IV | (isoform 1), |
| Erythropoietic | Ferrochelatase, P22830\|HEMH_HUMAN Ferrochelatase, mitochondrial, |
| Protoporphyria | (isoform 1), |
| Eczema | Filaggrin |
| Excess Fat | Thermogenin, P25874\|UCP1_HUMAN Mitochondrial brown fat uncoupling |
| | protein 1, |
| Excess Fat | Lipase; Lipoprotein lipase, P06858\|LIPL_HUMAN Lipoprotein lipase, Hepatic |
| | lipase, P11150\|LIPC_HUMAN Hepatic triacylglycerol lipase, Pancreatic lipase, |
| | P16233\|LIPP_HUMAN Pancreatic triacylglycerol lipase, Endothelial lipase, |
| | (isoform 1), Q9Y5X9\|LIPE_HUMAN Endothelial lipase, Lysosomal lipase, |
| | P38571\|LICH_HUMAN Lysosomal acid lipase/cholesteryl ester hydrolase, |
| | (isoform 1), (Hormone sensitive lipase, Q05469\|LIPS_HUMAN Hormone- |
| | sensitive lipas, (isoform 1), Gastric lipase, P07098\|LIPG_HUMAN Gastric |
| | triacylglycerol lipase, (isoform 1), Pancreatic Lipase-Related Protein 1), |
| | P54315\|LIPR1_HUMAN Inactive pancreatic lipase-related protein 1, (isoform |
| | 1),); Pancreatic Lipase-Related Protein 2, P54317\|LIPR2_HUMAN Pancreatic |
| | lipase-related protein 2, Carboxyl Ester Lipase, P19835\|CEL_HUMAN Bile |
| | salt-activated lipase, (isoform long), |
| Hypotrichosis | ADAM17, P78536\|ADA17_HUMAN Disintegrin and metalloproteinase |
| | domain-containing protein 17, (isoform A), |
| Ichthyosis Vulgaris | Filaggrin |
| Infections | Genetic Antibiotics (e.g. Anti-Sigma Factors) |
| Inflammatory and | Desmoglein 2, Q14126\|DSG2_HUMAN Desmoglein-2, |
| Bullous Skin Bowel | |
| Syndrome | |
| Keratosis Pilaris | Retinol Dehydrogenase 10 |
| Oily Skin | Retinol Dehydrogenase 10 |
| Osteoarthritis | Hyaluronan Synthase |
| Pemphigus Vulgaris | Plakophilin-1, Q13835\|PKP1_HUMAN Plakophilin-1, (isoform 2), |
| Pseudoxanthoma | Elastin, sp\|P15502\|ELN_HUMAN Elastin, (isoform 3), |
| elasticum | |
| Psoriasis | Retinol Dehydrogenase 10 |
| Scar Treatment | Tyrosinase, P14679\|TYRO_HUMAN Tyrosinase, (isoform 1), |
| Scarring | Elastin, sp\|P15502\|ELN_HUMAN Elastin, (isoform 3), |
| Scarring | Collagen Type I, P02452\|CO1A1_HUMAN Collagen alpha-1(I) chain, |
| | P08123\|CO1A2_HUMAN Collagen alpha-2(I) chain, |
| Scarring | Collagen Type III, P02461\|CO3A1_HUMAN Collagen alpha-1(III) chain, |
| | (isoform 1), |
| Skin Cancer | Interferon; Interferon, Alpha 1, P01562\|IFNA1_HUMAN Interferon alpha- |
| | 1/13, Interferon, Alpha 2, P01563\|IFNA2_HUMAN Interferon alpha-2, |
| | Interferon, Alpha 4, P05014\|IFNA4_HUMAN Interferon alpha-4, Interferon, |
| | Alpha 5, P01569\|IFNA5_HUMAN Interferon alpha-5, Interferon, Alpha 6, |
| | P05013\|IFNA6_HUMAN Interferon alpha-6, Interferon, Alpha 7, |
| | P01567\|IFNA7_HUMAN Interferon alpha-7, Interferon, Alpha 8, |
| | P32881\|IFNA8_HUMAN Interferon alpha-8, Interferon, Alpha 10, |
| | P01566\|IFN10_HUMAN Interferon alpha-10, Interferon, Alpha 14, |
| | P01570\|IFN14_HUMAN Interferon alpha-14 OS, Interferon, Alpha 16, |
| | P05015\|IFN16_HUMAN Interferon alpha-16, Interferon, Alpha 17, |
| | P01571\|IFN17_HUMAN Interferon alpha-17, Interferon, Alpha 21, |
| | P01568\|IFN21_HUMAN Interferon alpha-21, Interferon, Gamma, |
| | P01579\|IFNG_HUMAN Interferon gamma, Interferon, Beta, |
| | P01574\|IFNB_HUMAN Interferon beta, Interferon, Kappa, |
| | Q9P0W0\|IFNK_HUMAN Interferon kappa, Interferon, Epsilon, |
| | Q86WN2\|IFNE_HUMAN Interferon epsilon, |
| Striate Palmoplantar | ADAM17, P78536\|ADA17_HUMAN Disintegrin and metalloproteinase |
| Keratoderma | domain-containing protein 17, (isoform A), |
| Tanning | Tyrosinase, P14679\|TYRO_HUMAN Tyrosinase, (isoform 1), |
| Vitiligo | Melanocyte-Stimulating Hormone; Alpha-MSH, P01189\|138-150, Beta-MSH, |
| | P01189\|217-234, Gamma-MSH, P01189\|77-87, Proopiomelanocortin, |
| | P01189\|COLI_HUMAN Pro-opiomelanocortin, |
| Vitiligo | Tyrosinase, P14679\|TYRO_HUMAN Tyrosinase, (isoform 1), |
| Warts | Interferon; Interferon, Alpha 1, P01562\|IFNA1_HUMAN Interferon alpha- |
| | 1/13, Interferon, Alpha 2, P01563\|IFNA2_HUMAN Interferon alpha-2, |
| | Interferon, Alpha 4, P05014\|IFNA4_HUMAN Interferon alpha-4, Interferon, |
| | Alpha 5, P01569\|IFNA5_HUMAN Interferon alpha-5, Interferon, Alpha 6, |
| | P05013\|IFNA6_HUMAN Interferon alpha-6, Interferon, Alpha 7, |
| | P01567\|IFNA7_HUMAN Interferon alpha-7, Interferon, Alpha 8, |
| | P32881\|IFNA8_HUMAN Interferon alpha-8, Interferon, Alpha 10, |
| | P01566\|IFN10_HUMAN Interferon alpha-10, Interferon, Alpha 14, |
| | P01570\|IFN14_HUMAN Interferon alpha-14 OS, Interferon, Alpha 16, |
| | P05015\|IFN16_HUMAN Interferon alpha-16, Interferon, Alpha 17, |
| | P01571\|IFN17_HUMAN Interferon alpha-17, Interferon, Alpha 21, |
| | P01568\|IFN21_HUMAN Interferon alpha-21, Interferon, Gamma, |
| | P01579\|IFNG_HUMAN Interferon gamma, Interferon, Beta, |
| | P01574\|IFNB_HUMAN Interferon beta, Interferon, Kappa, |
| | Q9P0W0\|IFNK_HUMAN Interferon kappa, Interferon, Epsilon, |
| | Q86WN2\|IFNE_HUMAN Interferon epsilon, |
| Wound Healing | Elastin, sp\|P15502\|ELN_HUMAN Elastin, (isoform 3) |
| Wound Healing | Collagen Type I, P02452\|CO1A1_HUMAN Collagen alpha-1(I) chain, |
| | P08123\|CO1A2_HUMAN Collagen alpha-2(1) chain |
| Wound Healing | Collagen Type III, P02461\|CO3A1_HUMAN Collagen alpha-1(III) chain, |
| | (isoform 1) |
| Xeroderma | DNA Polymerase Eta, Q9Y253\|POLH_HUMAN DNA polymerase eta, |
| Pigmentosum | (isoform 1) |

TABLE 2
Illustrative Proteins and Illustrative Peptides. In Table 2, all Illustrative Identifiers (e.g.
Gene Seq nos. and references are hereby incorporated by reference in their entireties).
Protein/Peptide Illustrative Identifier Reference

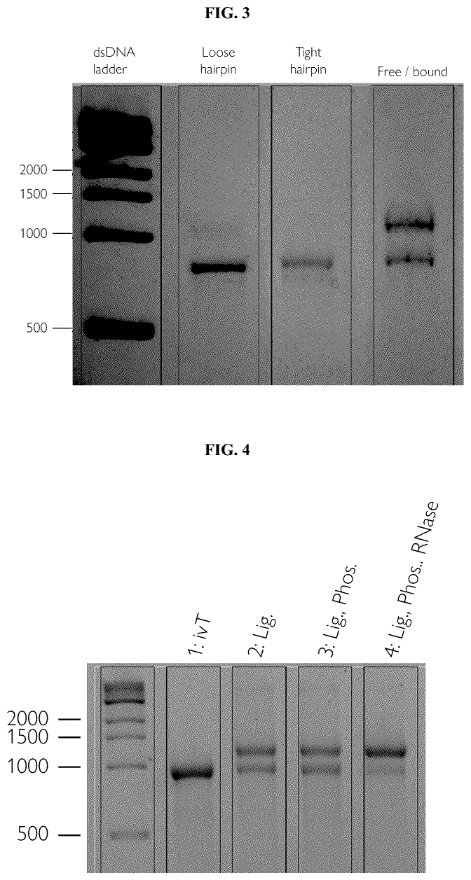

(DIL-40) alternative sequence GeneSeq Accession Y09197 WO9919491, SEQ ID NO: 4 of,
WO9919491,
4-1BB ligand GeneSeq Accession W26657 U.S. Pat. No. 5,674,704, P41273/TNFL9_HUMAN Tumor
necrosis factor ligand superfamily member 9
A human C-C chemokine designated exodus GeneSeq Accession B07939 U.S. Pat. No. 6,096,300,
P78556/CCL20_HUMAN C-C motif chemokine 20, (isoform 1)
A human L105 chemokine designated huL105_3. GeneSeq Accession W87588 WO9856818, SEQ ID
NO: 2 of WO9856818
A human L105 chemokine designated huL105_7. GeneSeq Accession W87589 WO9856818, SEQ ID
NO: 4 of WO9856818
A monokine induced by gamma- interferon (MIG) GeneSeq Accession W96710 U.S. Pat. No. 5,871,723,
Q07325/CXCL9_HUMAN C-X-C motif chemokine 9
A partial CXCR4B protein GeneSeq Accession W97363 EP897980, SEQ ID NO: 2 of EP897980
A platelet basic protein (PBP) GeneSeq Accession W96716 U.S. Pat. No. 5,871,723,
P02775/CXCL7_HUMAN Platelet basic protein
ACRP-30 Homologue; Complement Component Clq C GeneSeq Accession B30234 WO0063376,
P02747/C1QC_HUMAN Complement C1q subcomponent subunit C
Activator Inbibitor-1; PAI-1 GeneSeq Accession R08411 WO9013648, P05121/PAI1_HUMAN
Plasminogen activator inhibitor 1, (isoform 1)
Activator Inhibitor-2; PAI-2 GeneSeq Accession R10921 WO9102057, P05120/PAI2_HUMAN
Plasminogen activator inhibitor 2
Adenoid- expressed chemokine (ADEC) GeneSeq Accession R97664 WO9617868, SEQ ID NO: 2 of,
WO9617868
aFGF; FGF-1 GeneSeq Accession P94037 EP298723, P05230/FGF1_HUMAN Fibroblast growth factor
1, (isoform 1)
AGF GeneSeq Accession R64240 WO9429344, Q8NI99/ANGL6_HUMAN Angiopoietin-related
protein 6
AGF Protein GeneSeq Accession R92749 U.S. Pat. No. 5,488,032, Q8NI99/ANGL6_HUMAN
Angiopoietin-related protein 6,
ANGPTL3 Angiopoietin-like 3 GeneSeq Accession EAX06583.1
Antibodies specific for collapsin U.S. Pat. No. 5,416,197, Wildtype collapsin has the sequence:, SEQ ID
NO: 2 of 5,416,197
Anti-TGF beta family antibodies GB2305921
APM-I; ACRP- 30; Famoxin GeneSeq Accession Y71035 W00026363, Q15848/ADIPO_HUMAN
Adiponectin
b57 Protein GeneSeq Accession W69293 WO9837195, SEQ ID NO: 2 of WO9837195
BCMA GeneSeq Accession Y71979 WO0068378, Q02223/TNR17_HUMAN Tumor necrosis factor
receptor superfamily member 17, (isoform 1)
Beta-thrombo- globulin protein (beta-TG) GeneSeq Accession W96718 U.S. Pat. No. 5,871,723, SEQ ID
NO: 10 of U.S. Pat. No. 5,871,723
bFGF; FGF-2 GeneSeq Accession R06685 FR2642086, P09038/FGF2_HUMAN Fibroblast growth
factor 2, (isoform 1)
BMP Processing Enzyme Furin GeneSeq Accession W36099 WO9741250, P09958/FURIN_HUMAN
Furin
BMP-1 GeneSeq Accession P80618 WO8800205, P13497/BMP1_HUMAN Bone morphogenetic
protein 1, (isoform BMP1-3)
BMP-10 GeneSeq Accession R66202 WO9426893, O95393/BMP10_HUMAN Bone morphogenetic
protein 10
BMP-12 GeneSeq Accession R78734 WO9516035, Q7Z4P5/GDF7_HUMAN Growth/differentiation
factor 7
BMP-15 GeneSeq Accession W11261 W09636710, O95972/BMP15_HUMAN Bone morphogenetic
protein 15
BMP-17 GeneSeq Accession Y17870 WO9929718, SEQ ID NO: 2 from U.S. Pat. No. 7,151,086
BMP-18 GeneSeq Accession Y17871 WO9929718, SEQ ID NO: 4 from U.S. Pat. No. 7,151,086
BMP-2 GeneSeq Accession P80619 WO8800205, P12643/BMP2_HUMAN Bone morphogenetic
protein 2
BMP-2B GeneSeq Accession W24850 U.S. Pat. No. 5,631,142, P12644/BMP4_HUMAN Bone
morphogenetic protein 4
BMP-3, P12645|BMP3_HUMAN Bone morphogenetic protein 3
BMP-4 GeneSeq Accession B02796 WO0020591, P12644/BMP4_HUMAN Bone morphogenetic
protein 4
BMP-5 GeneSeq Accession B02797 WO0020591, P22003/BMP5_HUMAN Bone morphogenetic
protein 5, (isoform 1)
BMP-6 GeneSeq Accession R32904 U.S. Pat. No. 5,187,076, P22004/BMP6_HUMAN Bone
morphogenetic protein 6
BMP7 Variant A
BMP7 Variant B
BMP7 Variant C
BMP-9 GeneSeq Accession R86903 WO9533830, Q9UK05/GDF2_HUMAN Growth/differentiation
factor 2
Brain-Derived Neurotrophic Factor (BDNF)
Calpain-10a GeneSeq Accession Y79567 WO0023603, Q9HC96/CAN10_HUMAN Calpain-10,
(Isoform A)
Calpain-10b GeneSeq Accession Y79568 WO0023603, Q9HC96-2/CAN10_HUMAN Isoform B of
Calpain-10
Calpain-10c GeneSeq Accession Y79569 WO0023603, Q9HC96-3/CAN10_HUMAN Isoform C of
Calpain-10
Carboxypeptidase E, (CPE),
CASPASE1
C-C chemokine, MCP2 GeneSeq Accession Y05300 EP905240, P80075/CCL8_HUMAN C-C motif
chemokine 8
CCR5 variant GeneSeq Accession W88238 WO9854317, Variants of wildtype CCR5 which has the
sequence, of: P51681|CCR5_HUMAN C-C chemokine receptor type 5
CCR7 GeneSeq Accession B50859 U.S. Pat. No. 6,153,441, P32248/CCR7_HUMAN C-C chemokine
receptor type 7
CCR7 GeneSeq Accession B50859 U.S. Pat. No. 6,153,441, P32248/CCR7_HUMAN C-C chemokine
receptor type 7
CD137; 4-1BB Receptor Protein GeneSeq Accession R70977 WO9507984, Q07011/TNR9_HUMAN
Tumor necrosis factor receptor superfamily member 9
CD27 GeneSeq Accession R20814 WO9201049, P26842/CD27_HUMAN CD27 antigen
CD27L GeneSeq Accession R50121 WO9405691, P32970/CD70_HUMAN CD70 antigen, (isoform 1)
CD30 GeneSeq Accession R35478 DE4200043, P28908/TNR8_HUMAN Tumor necrosis factor
receptor superfamily member 8, (isoform 1)
CD30 ligand GeneSeq Accession R45007 WO9324135, P32971/TNFL8_HUMAN Tumor necrosis
factor ligand superfamily member 8
CD40 GeneSeq Accession Y33499 WO9945944, P25942/TNR5_HUMAN Tumor necrosis factor
receptor superfamily member 5, (isoform 1)
CD40L GeneSeq Accession R85486 WO9529935, P29965/CD40L_HUMAN CD40 ligand
Cerberus Protein GeneSeq Accession W86032 WO9849296, O95813/CER1_HUMAN Cerberus
Chemokine Eotaxin GeneSeq Accession Y14230 WO9912968, P51671/CCL11_HUMAN Eotaxin
Chemokine hIL-8 GeneSeq Accession Y14229 WO9912968, P10145/IL8_HUMAN Interleukin-8,
(isoform 1)
Chemokine hMCP1 GeneSeq Accession Y14222 WO9912968, P13500/CCL2_HUMAN C-C motif
chemokine 2
Chemokine hMCP1a GeneSeq Accession Y14225 WO9912968
Chemokine hMCP1b GeneSeq Accession Y14226 WO9912968
Chemokine hMCP2 GeneSeq Accession Y14223 WO9912968, P80075/CCL8_HUMAN C-C motif
chemokine 8
Chemokine hMCP3 GeneSeq Accession Y14224 WO9912968, P80098/CCL7_HUMAN C-C motif
chemokine 7
Chemokine hSDF1b GeneSeq Accession Y14228 WO9912968, P48061/SDF1_HUMAN Stromal cell-
derived factor 1, (isoform beta)
Chemokine MCP-4 GeneSeq Accession W56690 WO9809171, Q99616/CCL13_HUMAN C-C motif
chemokine 13
Chemokine-like protein IL-8M1 Full-Length and Mature GeneSeq Accessions R99815 and R99803
WO9613587, FIG. 4B of WO9613587
Chemokine-like protein IL-8M3 GeneSeq Accession R99812 WO9613587
Chemokine-like protein IL-8M8 Full-Length and Mature GeneSeq Accessions R99816 and R99805
WO9613587, FIG. 4C of WO9613587
Chemokine-like protein IL-8M8 Full-Length and Mature GeneSeq Accessions R99817 and R99806
WO9613587, FIG. 4C of WO9613587
Chemokine-like protein IL-8M8 Full-Length and Mature GeneSeq Accessions R99818 and R99804
WO9613587, FIG. 4C of WO9613587
Chemokine-like protein IL-8M8 Full-Length and Mature GeneSeq Accessions R99819 and R99807
WO9613587, FIG. 4C of WO9613587
Chemokine-like protein IL-8M8 Full-Length and Mature GeneSeq Accessions R99822 and R9807
WO9613587, FIG. 4C of WO9613587
Chemokine-like protein PF4-414 Full-Length and Mature GeneSeq Accessions R92318 and R99809
WO9613587, FIG. 3C of WO9613587
Chondro modulin-like protein GeneSeq Accession Y71262 WO0029579, SEQ ID NO: 2 from
WO0029579
c-kit ligand; SCF; Mast cell growth factor; MGF; Fibrosarcoma- derived stem cell factor GeneSeq
Accession Y53284, R83978 and R83977 EP992579 and EP676470, P21583|SCF_HUMAN Kit ligand,
(isoform 1)
Connective tissue activating protein-III (CTAP-III) GeneSeqAc- cession S96717 U.S. Pat. No. 5,871,723,
SEQ ID NO: 9 of U.S. Pat. No. 5,871,723
Constitutively active Akt (caAkt)
CTLA-8 GeneSeq Accession W13651 WO9704097, (also known as IL-17), Q16552/IL17_HUMAN
Interleukin-17A
CXC3 GeneSeq Accession W23345 WO9727299, P78423/X3CL1_HUMAN Fractalkine
CXC3 GeneSeq Accession W23345 WO9757599, P78423/X3CL1_HUMAN Fractalkine
CXCR3; CXC GeneSeq Accession Y79372 WO0018431, P49682|CXCR3_HUMAN C-X-C chemokine
receptor type 3, (isoform 1)
Dominant-negative TGF-β receptor II (dnTGF-βRII)
EDAR Genbank Accession AAD50077, Q9UNE0|EDAR_HUMAN Tumor necrosis factor receptor
superfamily member EDAR, (isoform 1)
Endostatin GeneSeq Accession B28399 WO0064946, P39060/COIA1_HUMAN Collagen alpha-
1(XVIII) chain, (isoform 1)
Endothelial Cell Specific Molecule 1, Gene ID: 11082
Eotaxin GeneSeq Accession W10099 WO9700960, P51671/CCL11_HUMAN Eotaxin
Epithelial neutrophil activating protein-78 (ENA-78) GeneSeq Accession W96712 U.S. Pat. No.
5,871,723, P42830/CXCL5_HUMAN C-X-C motif chemokine 5
EPO receptor; EPOR GeneSeq Accession R06512 WO9008822, P19235/EPOR_HUMAN
Erythropoietin receptor, (isoform EPOR-F)
Exodus protein GeneSeq Accession W61279 WO9821330, P78556/CCL20_HUMAN C-C motif
chemokine 20, (isoform 1)
FAS Ligand Inhibitory Protein (DcR3) GeneSeq Accession B19335 WO0058465,
O95407/TNF6B_HUMAN Tumor necrosis factor receptor superfamily member 6B
FasL GeneSeq Accession W95041 WO9903998, P48023/TNFL6_HUMAN Tumor necrosis factor
ligand superfamily member 6, (isoform 1)
FasL GeneSeq Accession W98071 WO9903999, P48023/TNFL6_HUMAN Tumor necrosis factor
ligand superfamily member 6, (isoform 1)
FasL GeneSeq Accession Y28594 WO9936079, P48023/TNFL6_HUMAN Tumor necrosis factor ligand
superfamily member 6, (isoform 1)
FGF-12; Fibroblast growth factor homologous factor-1 GeneSeq Accession W06309 WO9635708,
P61328/FGF12_HUMAN Fibroblast growth factor 12, (isoform 1)
FGF-16 GeneSeq Accession Y05474 WO9918128, O43320/FGF16_HUMAN Fibroblast growth factor
16
FGF-18 GeneSeq Accession Y08590 WO9927100, O76093/FGF18_HUMAN Fibroblast growth factor
18
FGF-19 GeneSeq Accession Y08582 WO9927100, O95750/FGF19_HUMAN Fibroblast growth factor
19
FGF-3; INT-2 GeneSeq Accession R07824 WO9503831, P11487/FGF3_HUMAN Fibroblast growth
factor 3
FGF-4; HST-1; HBGF-4 GeneSeq Accession R07825 WO9503831, P08620/FGF4_HUMAN Fibroblast
growth factor 4, (isoform 1)
FGF-5 GeneSeq Accession W22600 WO9730155, P12034/FGF5_HUMAN Fibroblast growth factor 5,
(isoform long)
FGF-6; Heparin binding secreted transforming factor-2 GeneSeq Accession R58555 EP613946,
P10767/FGF6_HUMAN Fibroblast growth factor 6
FGF-8 GeneSeq Accession R80783 WO9524928, P55075/FGF8_HUMAN Fibroblast growth factor 8,
(isoform 8E)
FGF-9; Gila activating factor GeneSeq Accession R70822 WO9503831, P31371/FGF9_HUMAN
Fibroblast growth factor 9
Fibroblast Growth Factor 1 (FGF1)
Fibroblast Growth Factor 1_vA, (FGF1_vA)
Fibroblast Growth Factor 1_vB, (FGF1_vB)
Fibroblast Growth Factor 1_vC, (FGF1_vC)
Fibroblast Growth Factor 19_vA, (FGF19_vA)
Fibroblast Growth Factor 21, (FGF21)
Fibroblast Growth Factor 23, (FGF23)
flt-1 GeneSeq Accession Y70751 WO0021560, P17948/VGFR1_HUMAN Vascular endothelial growth
factor receptor 1, (isoform 1)
flt-3 ligand GeneSeq Accession R67541 EP627487, P49771|FLT3L_HUMAN Fms-related tyrosine
kinase 3 ligand, (isoform 1)
Follicle stimulating hormone Alpha subunit GeneSeq Accession Y54160 EP974359,
P01215/GLHA_HUMAN Glycoprotein hormones alpha chain
Follicle stimulating hormone Beta subunit GeneSeq Accession Y54161 EP974359,
P01225/FSHB_HUMAN Follitropin subunit beta
Fractalkine GeneSeq Accession Y53255 U.S. Pat. No. 6,043,086, P78423/X3CL1_HUMAN Fractalkine
Fragment. myofibrillar protein troponin I GeneSeq Accession W18053 W09719955, SEQ ID NO: 3 of
WO9719955
Galectin-4 GeneSeq Accession W11841 WO9703190, P56470/LEG4_HUMAN Galectin-4
GDF-1 GeneSeq Accession R60961 WO9406449, P27539/GDF1_HUMAN Embryonic
growth/differentiation factor 1
Glioma-derived growth factor GeneSeq Accession R08120 EP399816
Glycodelin-A; Progesterone- associated endometrial protein GeneSeq Accession W00289 WO9628169,
P09466/PAEP_HUMAN Glycodelin
Granulocyte chemotactic protein-2 (GCP-2) GeneSeq Accession W96720 U.S. Pat. No. 5,871,723,
P80162/CXCL6_HUMAN C-X-C motif chemokine 6
Growth related oncogene- gamma (GRO-gamma) GeneSeq Accession W96715 U.S. Pat. No. 5,871,723,
P19876/CXCL3_HUMAN C-X-C motif chemokine 3
Growth related oncogene-alpha (GRO-alpha). GeneSeq Accession W96713 U.S. Pat. No. 5,871,723,
P09341/GROA_HUMAN Growth-regulated alpha protein
Growth related oncogene-beta (GRO-beta). GeneSeq Accession W96714 U.S. Pat. No. 5,871,723,
P19875/CXCL2_HUMAN C-X-C motif chemokine 2
hAPO-4; TROY GeneSeq Accession W93581 WO9911791, Q9NS68/TNR19_HUMAN Tumor necrosis
factor receptor superfamily member 19, (isoform 1)
HIV Inhibitor Peptide GeneSeq Accession Y89687 WO9959615
HIV Inhibitor Peptide GenSeq Accession Y31955 WO9948513
HIV Inhibitor Peptide Science 291, 884 (2001); Published online 12 Jan. 2001; 10.1126/science.1 057453
HIV protease inhibiting peptides GeneSeq Accessions R59293, R59294, R59295, R59296, R59297,
R59298, R59299, R592300, R59301, R59302, R59301, R59302, R59303, R59304, R59305, R59306,
R59307, R59308, R59309, R59310, R59311, R59312, R59313, R59314, R59315, R59316, R59317
R59318, R59319, R59320, R59321, R59322, R59323, R59324, R59325, R59326, R59327, R59328,
R59329, R59330, R59331, R59332, R59333, R59334, R59335, R59336, R59337, R59338, R59339,
R59340, R59341, R59342, R59343, R59344, R59345, R59346, R59347, R59348, R59349, and R59350
WO9301828
HIV-1 protease inhibitors GeneSeq Accessions R86326, R86327, R86328, R86329, R86330, R86331,
R86332, R86333, R86334, R86335, R86336, R86337, R86338, R86339, R86340, R86341, R86342,
R86343, R86344, R86345, R86346, R86347, R86348, R86349, R86350, R86351, R86352, R86353,
R86354, R86355, R86356, R86357, R86358, R86359, R86360, R86361, R86362, R86363, R86364,
R86365, R86366, R86367, R86368, R86369, R86370, and R86371 DE4412174
Human 6CKine protein GeneSeq Accession W50885 W09814581, SEQ ID NO: 8 of WO9814581,
Human Act-2 protein GeneSeq Accession W82717 WO9854326, P13236/CCL4_HUMAN C-C motif
chemokine 4
Human adipogenesis inhibitory factor GeneSeq Accession R43260 EP566410, (also known as IL-11),
P2080/|IL11_HUMAN Interleukin-11, (isoform 1),
Human B cell stimulating factor-2 receptor GeneSeq Accession P90525 AU8928720,
P08887/IL6RA_HUMAN Interleukin-6 receptor subunit alpha, (isoform 1)
Human beta-chemokine H1305 (MCP-2) GeneSeq Accession W26655 WO9725427,
P80075/CCL8_HUMAN C-C motif chemokine 8
Human C-C chemokine DGWCC GeneSeq Accession W60650 WO9823750, SEQ ID NO: 6 of
WO9823750
Human CC chemokine ELC protein GeneSeq Accession W62542 WO9826071,
Q99731/CCL19_HUMAN C-C motif chemokine 19
Human CC type chemokine interleukin C GeneSeq Accession Y57771 JP11302298
Human CCC3 protein GeneSeq Accession W82723 WO9854326, SEQ ID NO: 39 of WO9854326
Human CCF18 chemokine GeneSeq Accession W25942 WO9721812, SEQ ID NO: 4 of WO9721812
Human CC-type chemokine protein designated SLC (secondary lymphoid chemokine) GeneSeq Accession
W69163 WO9831809, O00585/CCL21_HUMAN C-C motif chemokine 21
Human chemokine beta-8 short forms GeneSeq Accession W16315 WO9712041, Wildtype chemokine
beta-8 provided as:, P55773|CCL23_HUMAN C-C motif chemokine 23
Human chemokine C10 GeneSeq Accession B15807 WO0042071, SEQ ID NO: 49 of WO0042071
Human chemokine CC- 3 GeneSeq Accession W38172 WO9741230, Q16627/CCL14_HUMAN C-C
motif chemokine 14
Human chemokine CC-2 GeneSeq Accession W38170 WO9741230, Q16663/CCL15_HUMAN C-C
motif chemokine 15
Human chemokine CCR-2 GeneSeq Accession B15808 WO0042071, P41597/CCR2_HUMAN C-C
chemokine receptor type 2, (isoform A)
Human chemokine Ckbeta-7 GeneSeq Accession Y96280 WO0028035, FIG. 1 of WO0028035
Human chemokine ENA-78 GeneSeq Accession B15809 WO0042071, P42830/CXCL5_HUMAN C-X-
C motif chemokine 5
Human chemokine eotaxin GeneSeq Accession B15794 WO0042071, P51671/CCL11_HUMAN
Eotaxin
Human chemokine GRO alpha GeneSeq Accession B15813 WO0042071, P09341/GROA_HUMAN
Growth-regulated alpha protein
Human chemokine GRO-alpha GeneSeq Accession B15793 WO0042071, P09341/GROA_HUMAN
Growth-regulated alpha protein
Human chemokine GRObeta GeneSeq Accession B15810 WO0042071, P19875/CXCL2_HUMAN C-
X-C motif chemokine 2
Human chemokine HCC-1 GeneSeq Accession B15806 WO0042071, Q16627/CCL14_HUMAN C-C
motif chemokine 14
Human chemokine HCC-1 GeneSeq Accession W38171 WO9741230, Q16627/CCL14_HUMAN C-C
motif chemokine 14
Human chemokine I- 309 GeneSeq Accession B15805 WO0042071, P22362/CCL1_HUMAN C-C motif
chemokine 1
Human chemokine IP-10 GeneSeq Accession B15811 WO0042071, P02778/CXL10_HUMAN C-X-C
motif chemokine 10
Human chemokine L105_3 GeneSeq Accession Y96923 U.S. Pat. No. 6,084,071, SEQ ID NO: 2 of
WO9856818
Human chemokine L105_7 GeneSeq Accession Y96922 U.S. Pat. No. 6,084,071, SEQ ID NO: 4 of
WO9856818
Human chemokine MIG GeneSeq Accession B15803 WO0042071, Q07325/CXCL9_HUMAN C-X-C
motif chemokine 9
Human chemokine MIG-beta protein GeneSeq Accession W90124 EP887409
Human chemokine MIP-1alpha GeneSeq Accession Y96281 WO0028035, P10147/CCL3_HUMAN C-
C motif chemokine 3,
Human chemokine MIP1beta GeneSeq Accession B15831 WO0042071, P13236/CCL4_HUMAN C-C
motif chemokine 4
Human chemokine MIP-3alpha GeneSeq Accession W44398 WO9801557, P78556/CCL20_HUMAN
C-C motif chemokine 20, (isoform 1)
Human chemokine MIP-3beta GeneSeq Accession W44399 WO9801557, Q99731/CCL19_HUMAN C-
C motif chemokine 19
Human chemokine PF4 GeneSeq Accession B15804 WO0042071, P02776/PLF4_HUMAN Platelet
factor 4
Human chemokine protein 331D5 GeneSeq Accession W59433 WO9811226, SEQ ID NO: 12 of
WO9811226
Human chemokine protein 61164 GeneSeq Accession W59430 WO9811226, SEQ ID NO: 6 of
WO9811226
Human chemokine receptor CXCR3 GeneSeq Accession Y79372 WO0018431,
P49682|CXCR3_HUMAN C-X-C chemokine receptor type 3, (isoform 1)
Human chemokine SDF1alpha GeneSeq Accession B15791 WO0042071, P48061-2/SDF1_HUMAN
Isoform Alpha of Stromal cell-derived factor 1, (isoform alpha)
Human chemokine SDF1beta GeneSeq Accession B15812 WO0042071, P48061/SDF1_HUMAN
Stromal cell-derived factor 1, (isoform beta)
Human chemokine ZSIG-35 GeneSeq Accession W30565 WO9844117, SEQ ID NO: 2 of WO
WO9844117
Human Chr19kine protein GeneSeq Acession W50887 WO9814581, SEQ ID NO: 10 of WO9814581
Human CKbeta- 9 GeneSeq Accession B50860 U.S. Pat. No. 6,153,441, O00585/CCL21_HUMAN C-C
motif chemokine 21
Human CKbeta-9 GeneSeq Accession B50860 U.S. Pat. No. 6,153,441, SEQ ID NO: 2 of U.S. Pat. No.
6,153,441
Human CX3C 111 amino acid chemokine GeneSeq Accession W23344 WO9727299, SEQ ID NO: 2 of
WO9727299
Human DNAX interleukin-40 GeneSeq Accession Y09196 WO9919491, SEQ ID NO: 2 or 4 of,
WO9919491,
Human DVic-1 C-C chemokine GeneSeq Accession W60649 WO9823750, SEQ ID NO: 2 of
WO9823750
Human EDIRF I protein sequence GeneSeq Accession Y22197 WO9932632, SEQ ID NO: 2 of
WO9932632
Human EDIRF II protein sequence GeneSeq Accession Y22199 WO9932632, SEQ ID NO: 6 of
WO9932632
Human eosinocyte CC type chemokine eotaxin GeneSeq Accession W14990 WO9712914,
P51671/CCL11_HUMAN Eotaxin
Human eosinophil- expressed chemokine (EEC) GeneSeq Accession W05186 WO9632481, SEQ ID
NO: 2 of WO9632481
Human fast twitch skeletal muscle troponin C GeneSeq Accession W22597 W09730085,
P02585/TNNC2_HUMAN Troponin C, skeletal muscle
Human fast twitch skeletal muscle troponin I GeneSeq Accession W18054 W09730085,
P48788/TNNI2_HUMAN Troponin I, fast skeletal muscle, (isoform 1)
Human fast twitch skeletal muscle Troponin subunit C GeneSeq Accession B00134 WO0054770, SEQ
ID NO: 1 of WO0054770
Human fast twitch skeletal muscle Troponin subunit I Protein GeneSeq Accession B00135 WO0054770,
SEQ ID NO: 2 of WO0054770
Human fast twitch skeletal muscle Troponin subunit T GeneSeq Accession B00136 WO0054770, SEQ
ID NO: 3 of WO0054770
Human fast twitch skeletal muscle troponin T GeneSeq Accession W22599 W09730085, SEQ ID NO: 3
of WO9730085
Human foetal spleen ex- pressed chemo-kine, FSEC GeneSeq Accession R98499 WO9622374, SEQ ID
NO: 2 of, WO9622374
Human GM- CSF receptor GeneSeq Accession R10919 WO9102063, GM-CSF receptor A,
P15509/CSF2R_HUMAN Granulocyte-macrophage colony-stimulating factor receptor subunit alpha,
(isoform 1), GM-CSF receptor B, P32927/IL3RB_HUMAN Cytokine receptor common subunit beta,
(isoform 1)
Human gro-alpha chemokine GeneSeq Accessions R66698 and W18024 WO9429341,
P09341/GROA_HUMAN Growth-regulated alpha protein
Human gro-beta chemokine GeneSeq Accessions R66699 and W17671 WO9429341,
P19875/CXCL2_HUMAN C-X-C motif chemokine 2
Human gro-gamma chemokine GeneSeq Accessions R66700 and W17672 WO9429341,
P19876/CXCL3_HUMAN C-X-C motif chemokine 3
Human IL- 1RD10 protein sequence GeneSeq Accession Y14131 WO9919480, SEQ ID NO: 20 of
WO9919480
Human IL- 1RD9 GeneSeq Accession Y14122 WO9919480, SEQ ID NOS: 6, 8, 10 of WO9919480
Human IL-16 protein GeneSeq Accession W33234 DE19617202, Q14005/IL16_HUMAN Pro-
interleukin-16, (isoform 1)
Human IL-5 receptor alpha chain GeneSeq Accession R25064 EP492214, Q01344/IL5RA_HUMAN
Interleukin-5 receptor subunit alpha, (isoform 1)
Human IL-6 receptor GeneSeq Accession Y30938 JP11196867, P08887/IL6RA_HUMAN Interleukin-
6 receptor subunit alpha, (isoform 1)
Human IL-8 receptor protein hIL8R GeneSeq Accession B09990 JP08103276, IL-8RA,
P25024/CXCR1_HUMAN C-X-C chemokine receptor type 1, IL-8RB, P25025/CXCR2_HUMAN C-
X-C chemokine receptor type 2
Human IL-8 receptor protein hIL8RA GeneSeq Accession B09989 JP08103276, IL-8RA,
P25024/CXCR1_HUMAN C-X-C chemokine receptor type 1
Human IL-9 receptor protein GeneSeq Accessions W64055, W64056, and W64057 WO9824904,
Q01113/IL9R_HUMAN Interleukin-9 receptor, (isoform 1)
Human IL-9 receptor protein variant #3. GeneSeq Accession W64058 WO9824904, Wildtype IL-9R is
provided as:, Q01113/IL9R_HUMAN Interleukin-9 receptor, (isoform 1)
Human IL-9 receptor protein variant #3. GeneSeq Accession W64061 WO9824904, Wildtype IL-9R is
provided as:, Q01113/IL9R_HUMAN Interleukin-9 receptor, (isoform 1)
Human IL-9 receptor protein variant fragment GenSeq Accession W64060 WO9824904, Wildtype IL-9R
is provided as:, Q01113/IL9R_HUMAN Interleukin-9 receptor, (isoform 1)
Human interlaukin-12 40 kD subunit GeneSeq Accession R79187 WO9519786,
P2946/|IL12B_HUMAN Interleukin-12 subunit beta
Human interleukin 1 delta GeneSeq Accession Y28408 WO9935268, SEQ ID NO: 4 of WO9935268
Human interleukin 18 derivatives GeneSeq Accessions W77083, W77084, W77085, W77086, W77087,
W77088, and W77089 EP861663, Variants of wildtype IL18 which is provided as:,
Q14116/IL18_HUMAN Interleukin-18, (isoform 1)
Human Interleukin-1 beta precursor GeneSeq Accession R42447 EP569042, P01584/IL1B_HUMAN
Interleukin-1 beta
Human interleukin-1 beta precursor. GeneSeq Accession R42447 EP569042, P01584/IL1B_HUMAN
Interleukin-1 beta
Human interleukin-1 receptor accessory protein GeneSeq Accession W01911 WO9623067, Human
IL1R Acp, SEQ ID NO: 3 of WO9623067, Soluble Human IL1R Acp, SEQ ID NO: 9 of WO9623067
Human interleukin-1 receptor antagonist beta GeneSeq Accession Y24395 WO9935268,
Human interleukin-1 type-3 receptor GeneSeq Accession R91064 W09607739, SEQ ID NO: 2 and 4 of
WO9607739,
Human Interleukin-10 (precursor) GeneSeq Accession R41664 WO9317698, P22301/IL10_HUMAN
Interleukin-10, (precursor form is processed into a truncated mature form)
Human Interleukin-10 (precursor) GeneSeq Accession R41664 WO9317698, P22301/IL10_HUMAN
Interleukin-10, (precursor form is processed into a truncated mature form)
Human Interleukin-10 GeneSeq Accession R42642 WO9318783, SEQ ID NO: 3 of WO9318783-A,
(mature IL-10)
Human Interleukin-10 GeneSeq Accession R42642 WO9318783-A, SEQ ID NO: 3 of WO9318783-A,
(mature IL-10)
Human interleukin-11 receptor GeneSeq Accession R99090 WO9619574, Q14626/I11RA_HUMAN
Interleukin-11 receptor subunit alpha
Human interleukin-12 beta-1 receptor. GeneSeq Accession W12772 EP759466, P4270/|I12R1_HUMAN
Interleukin-12 receptor subunit beta-1, (isoform 1)
Human interleukin-12 beta-2 receptor GeneSeq Accession W12771 EP759466, Q9966/|I12R2_HUMAN
Interleukin-12 receptor subunit beta-2, (isoform 1)
Human Interleukin-12 p35 protein GeneSeq Accession W51312 WO9817689, P29459/IL12A_HUMAN
Interleukin-12 subunit alpha
Human Interleukin-12 p40 protein GeneSeq Accession W51311 WO9817689, P2946/|IL12B_HUMAN
Interleukin-12 subunit beta
Human interleukin-12 receptor GeneSeq Accession R69632 EP638644, IL-12 receptor B1,
P42701|I12R1_HUMAN Interleukin-12 receptor subunit beta-1, (isoform 1), IL-12 receptor B2,
Q99665|I12R2_HUMAN Interleukin-12 receptor subunit beta-2, (isoform 1)
Human interleukin-13 alpha receptor GeneSeq Accession W24973 WO9720926, IL-13RA1,
P78552/I13R1_HUMAN Interleukin-13 receptor subunit alpha-1, (isoform 1), IL-13RA2,
Q14627/I13R2_HUMAN Interleukin-13 receptor subunit alpha-2,
Human interleukin-13 beta receptor GeneSeq Accession W24972 WO9720926, SEQ ID NO: 2 from
WO9720926
Human interleukin-15 GeneSeq Accession W53878 U.S. Pat. No. 5,747,024, P40933/IL15_HUMAN
Interleukin-15, (isoform IL15-S48AA)
Human interleukin-15 receptor from clone P1 GeneSeq Accession R90843 WO9530695,
Q13261|I15RA_HUMAN Interleukin-15 receptor subunit alpha, Isoform 1)
Human interleukin-17 receptor GeneSeq Accession W04185 WO9629408, Q96F46/I17RA_HUMAN
Interleukin-17 receptor A
Human interleukin-18 GeneSeq Accession W77077 EP861663, Q14116/IL18_HUMAN Interleukin-18,
(isoform 1)
Human interleukin-18 protein (IL-18) GeneSeq Accession W77158 EP864585, Q14116/IL18_HUMAN
Interleukin-18, (isoform 1)
Human interleukin-3 receptor GeneSeq Accession R25300 EP509826, P26951/IL3RA_HUMAN
Interleukin-3 receptor subunit alpha, (isoform 1)
Human interleukin-3 variant GeneSeq Accession R22814 JP04063595, Variants of wildtype IL-3 which
has the sequence:, P08700|IL3_HUMAN Interleukin-3
Human interleukin-4 receptor GeneSeq Accession W13499 U.S. Pat. No. 5,599,905,
P24394/IL4RA_HUMAN Interleukin-4 receptor subunit alpha, (isoform 1)
Human interleukin-5 GeneSeq Accession R92802 WO9604306, P05113/IL5_HUMAN Interleukin-5
Human interleukin-6 GeneSeq Accession R49041 WO9403492, P05231/IL6_HUMAN Interleukin-6
Human interleukin-7 GeneSeq Accession R59919 U.S. Pat. No. 5,328,988, P13232/IL7_HUMAN
Interleukin-7, (isoform 1)
Human interleukin-7 GeneSeq Accession R92796 WO9604306, P13232/IL7_HUMAN Interleukin-7,
(isoform 1)
Human interleukin-8 (IL-8) GeneSeq Accession R99814 WO9613587, P10145/IL8_HUMAN
Interleukin-8, (isoform 1)
Human intracellular IL- 1 receptor antagonist. GeneSeq Accession W77158 EP864585 (e.g. SEQ ID
NOs:12 to 19, or 22 to 25 of this publication).
Human IP-10 and HIV-1 gp 120 hyper- variable region fusion protein GeneSeq Accession Y29897
W09946392, Wildtype IP10 has the sequence:, P02778/CXL10_HUMAN C-X-C motif chemokine 10,
Wildtype gp120 has the sequence:, P03378|32-509, (cleaved product of gp160)
Human IP-10 and human Muc-1 core epitope (VNT) fusion protein GeneSeq Accession Y29894
WO9946392, Wildtype IP10 has the sequence:, P02778/CXL10_HUMAN C-X-C motif chemokine 10,
Wildtype Muc-1 has the sequence:, P15941|MUC1_HUMAN Mucin-1, (isoform 1)
human liver and activation regulated chemokine (LARC) GeneSeq Accession W57475 WO9817800,
P78556/CCL20_HUMAN C-C motif chemokine 20, (isoform 1)
Human Lkn-1 Full-Length and Mature protein GeneSeq Accessions Y17280, Y17274, Y17281, and
Y17275 WO9928473 and WO9928472, Q16663/CCL15_HUMAN C-C motif chemokine 15
Human mammary associated chemokine (MACK) protein Full-Length and Mature GeneSeq Accessions
Y29092 and Y29093 WO9936540, Full-length: SEQ ID NO: 1 of WO9936540, Mature Form: SEQ ID
NO: 2 of WO9936540
Human mature chemokine Ckbeta-7 (optionally truncated) GenSeq Accession Y96282 WO0028035,
FIG. 1 of WO0028035,
Human mature gro-alpha polypeptide used to treat sepsis GeneSeq Accession W81498 WO9848828,
P09341/GROA_HUMAN Growth-regulated alpha protein
Human mature gro-gamma polypeptide used to treat sepsis GeneSeq Accession W81500 WO9848828,
P19876/CXCL3_HUMAN C-X-C motif chemokine 3
Human MCP-3 and human Muc-1 core epitope (VNT) fusion protein GeneSeq Accession Y29893
WO9946392, Wildtype MCP-3 has the sequence:, P80098/CCL7_HUMAN C-C motif chemokine 7,
Wildtype Muc-1 has the sequence:, P15941|MUC1_HUMAN Mucin-1, (isoform 1)
Human MI10 protein GeneSeq Accession W82721 WO9854326, SEQ ID NO: 37 of WO9854326
Human MI1A protein GeneSeq Accession W82722 W09854326, SEQ ID NO: 38 of WO9854326
Human monocyte chemoattractant factor hMCP-1 GeneSeq Accession R73914 WO9509232,
P13500/CCL2_HUMAN C-C motif chemokine 2
Human monocyte chemoattractant factor hMCP-3 GeneSeq Accession R73915 WO9509232,
P80098/CCL7_HUMAN C-C motif chemokine 7
Human monocyte chemotactic proprotein (MCPP) sequence GeneSeq Accession W42072 WO9802459,
SEQ ID NO: 1 of WO9802459
Human neurotactin chemokine like domain GeneSeq Accession Y53259 U.S. Pat. No. 6,043,086,
P78423/X3CL1_HUMAN Fractalkine
Human non- ELR CXC chemokine H174 GeneSeq Accession Y96310 WO0029439,
O14625/CXL11_HUMAN C-X-C motif chemokine 11
Human non- ELR CXC chemokine IP10 GeneSeq Accession Y96311 WO0029439,
P02778/CXL10_HUMAN C-X-C motif chemokine 10
Human non- ELR CXC chemokine Mig GeneSeq Accession Y96313 WO0029439,
Q07325/CXCL9_HUMAN C-X-C motif chemokine 9
Human PAI-1 mutants GeneSeq Accessions R11755, R11756, R11757, R11758, R11759, R11760,
R11761, R11762 and R11763 WO9105048, Wildtype PAI-1 is provided as P05121/PAI1_HUMAN
Plasminogen activator inhibitor 1, (isoform 1)
Human protein with IL-16 activity GeneSeq Accession W59425 DE19649233-
Human protein with IL-16 activity GeneSeq Accession W63753 DE19649233-
Human secondary lymphoid chemokine (SLC) GeneSeq Accession B01434 WO0038706,
O00585/CCL21_HUMAN C-C motif chemokine 21
Human SISD protein GeneSeq Accession W82720 WO9854326, P13501/CCL5_HUMAN C-C motif
chemokine 5
Human STCP-1 GeneSeq Accession W62783 WO9824907, O00626/CCL22_HUMAN C-C motif
chemokine 22
Human stromal cell-derived chemokine, SDF-1 GeneSeq Accession W50766 FR2751658,
P48061/SDF1_HUMAN Stromal cell-derived factor 1, (isoform beta)
Human T cell mixed lymphocyte reaction expressed chemokine (TMEC) GeneSeq Accession W58703
U.S. Pat. No. 5,780,268, SEQ ID NO: 2 of U.S. Pat. No. 5,780,268
Human thymus and activation regulated cytokine (TARC) GeneSeq Accession W14018 WO9711969,
Q92583/CCL17_HUMAN C-C motif chemokine 17
Human thymus expressed chemokine TECK and TECK variant GeneSeq Accessions B19607 and
B19608 WO0053635, Wildtype TECK provided as: O15444/CCL25_HUMAN C-C motif chemokine
25
Human TNF- alpha GeneSeq Accession R42679 EP563714, P01375/TNFA_HUMAN Tumor necrosis
factor
Human TNF- alpha GeneSeq Accession R62463 EP619372, P01375/TNFA_HUMAN Tumor necrosis
factor
Human TNF- beta (LT-alpha) GeneSeq Accession B37799 WO0064479, P01374/TNFB_HUMAN
Lymphotoxin-alpha
Human type CC chemokine eotaxin 3 protein sequence GeneSeq Accession Y43178 JP11243960,
Q9Y258/CCL26_HUMAN C-C motif chemokine 26
Human type II interleukin-1 receptor GeneSeq Accession R85480 U.S. Pat. No. 5,464,937,
P27930/IL1R2_HUMAN Interleukin-1 receptor type 2
Human wild- type interleukin-4 (hIL-4) protein GeneSeq Accession W52149 WO9747744,
P05112/IL4_HUMAN Interleukin-4, (isoform 1)
Human ZCHEMO-8 GeneSeq Accession W82716 WO9854326, SEQ ID NO: 2 of WO9854326
Humanized Anti-VEGF Antibodies, and fragments thereof WO0029584
Humanized Anti-VEGF Antibodies, and fragments thereof WO9845331
ICE 10 kD subunit GeneSeq Accession R33782 EP533350, SEQ ID NO: 18 of EP533350
ICE 20 kD subunit. GeneSeq Accession R33781 EP533350, SEQ ID NO: 17 of EP533350
ICE 22 kD subunit. GeneSeq Accession R33780 EP533350, SEQ ID NO: 16 of EP533350
IL-1 alpha GeneSeq Accession P90108 EP324447, P01583|IL1A_HUMAN Interleukin-1 alpha
IL-1 beta GeneSeq Accession Y08322 WO9922763, P01584|IL1B_HUMAN Interleukin-1 beta
IL-1 GeneSeq Accession P60326 EP165654, IL-1 alpha, P01583|IL1A_HUMAN Interleukin-1 alpha,
IL-1 beta, P01584|IL1B_HUMAN Interleukin-1 beta
IL-1 inhibitor (IL-li) GeneSeq Accession R35486 and R35484 EPS541920
IL-1 mature GeneSeq Accession R14855 EP456332, (mature truncated form wherein the precursor is
cleaved between amino acids 116-117)
IL-10 receptor GeneSeq Accession W41804 U.S. Pat. No. 5,716,804, IL-10RA, Q13651/I10R1_HUMAN
Interleukin-10 receptor subunit alpha, IL-10RB, Q0833/|I10R2_HUMAN Interleukin-10 receptor
subunit beta
IL-11 GeneSeq Accession R50176 WO9405318, P2080/|IL11_HUMAN Interleukin-11, (isoform 1),
IL-11 GeneSeq Accession W02202 JP08127539, P2080/|IL11_HUMAN Interleukin-11, (isoform 1)
IL-12 p40 subunit. GeneSeq Accession R63018 AU9466072, P2946/|IL12B_HUMAN Interleukin-12
subunit beta
IL-13 GeneSeq Accession R48624 WO9404680, P35225/IL13_HUMAN Interleukin-13
IL-14 GeneSeq Accession R55800 WO9416074, P40222/TXLNA_HUMAN Alpha-taxilin
IL-15 receptor GeneSeq Accession R90843 WO9530695, Q1326/|I15RA_HUMAN Interleukin-15
receptor subunit alpha, (isoform 1)
IL-17 GeneSeq Accession R76573 WO9518826, Q16552/IL17_HUMAN Interleukin-17A
IL-17 receptor GeneSeq Accession B03807 U.S. Pat. No. 6,072,033, Q96F46/I17RA_HUMAN
Interleukin-17 receptor A
Il-17 receptor GeneSeq Accession Y97131 U.S. Pat. No. 6,100,235, Q96F46/I17RA_HUMAN
Interleukin-17 receptor A
Il-17 receptor GeneSeq Accession Y97181 U.S. Pat. No. 6,096,305, Q96F46/I17RA_HUMAN
Interleukin-17 receptor A
IL-19 GeneSeq Accession W37935 WO9808870, Q9UHD0|IL19_HUMAN Interleukin-19, (isoform 1)
IL-1i fragments GeneSeq Accession R35484 and R35485 EP541920
IL-21 (TIF) GeneSeq Accession Y92879 WO0024758, Q9HBE4/IL21_HUMAN Interleukin-21,
(isoform 1)
IL-3 containing fusion protein. GeneSeq Accession R79342 and R79344 WO9521254, Fusions of
wildtype IL-3 which has the sequence:, P08700|IL3_HUMAN Interleukin-3
IL-3 mutant proteins GeneSeq Accession R79254, R79255, R79256, R79257, R79258, R79259, R79260,
R79261, R79262, R79263, R79264, R79265, R79266, R79267, R79268, R79269, R79270, R79271,
R79272, R79273, R79274, R79275, R79276, R79277, R79278, R79279, R79280, R79281, R79282,
R79283, R79284, and R79285 ZA9402636, Variants of wildtype IL-3 which has the sequence:,
P08700|IL3_HUMAN Interleukin-3
IL-3 variants GeneSeq Accession P80382, P80383, P80384, and P80381 WO8806161, Variants of
wildtype IL-3 which has the sequence:, P08700|IL3_HUMAN Interleukin-3
IL-3 variants GeneSeq Accession R38561, R38562, R38563, R38564, R38565, R38566, R38567,
R38568, R38569, R38570, R38571, and R38572 WO9307171, Variants of wildtype IL-3 which has the
sequence:, P08700|IL3_HUMAN Interleukin-3
IL-4 GeneSeq Accession P70615 WO8702990, P05112/IL4_HUMAN Interleukin-4, (isoform 1)
IL-4 mutein GeneSeq Accession R47182 DE4137333, Variants of wildtype IL-4 which has the
sequence:, P05112/IL4_HUMAN Interleukin-4, (isoform 1
IL-4 mutein Y124G GeneSeq Accession R47184 DE4137333, Variants of wildtype IL-4 which has the
sequence:, P05112/IL4_HUMAN Interleukin-4, (isoform 1)
IL-4 mutein Y124X GeneSeq Accession R47183 DE4137333, Variants of wildtype IL-4 which has the
sequence:, P05112/IL4_HUMAN Interleukin-4, (isoform 1)
IL-4 muteins GeneSeq Accession W52151 W52152 W52153 W52154 W52155 W52156 W52157
W52158 W52159 W52160 W52161 W52162 W52163 W52164 and W52165 WO9747744, Variants of
wildtype IL-4 which has the sequence:, P05112/IL4_HUMAN Interleukin-4, (isoform 1)
Il-5 receptor GeneSeq Accession W82842 WO9847923, Q01344/IL5RA_HUMAN Interleukin-5
receptor subunit alpha, (isoform 1)
IL-6 GeneSeq Accession R45717 and R45718 WO9402512, P05231/IL6_HUMAN Interleukin-6
Il-6 receptor GeneSeq Accession R37215 JP05091892, P08887/IL6RA_HUMAN Interleukin-6 receptor
subunit alpha, (isoform 1)
IL-7 receptor clone GeneSeq Accession R08330 EP403114, P1687/|IL7RA_HUMAN Interleukin-7
receptor subunit alpha, (isoform 1)
IL-8 receptor GeneSeq Accession R33420 WO9306229, IL-8RA, P25024/CXCR1_HUMAN C-X-C
chemokine receptor type 1, IL-8RB, P25025/CXCR2_HUMAN C-X-C chemokine receptor type 2
IL-9 mature protein variant (Met117 version) GenSeq Accession W68157 WO9827997, FIG. 3 of
WO9827997
Inhibin alpha GeneSeq Accession B02806 WO0020591, P05111/INHA_HUMAN Inhibin alpha chain
Inhibin beta GeneSeq Accession H02808 WO0020591, P08476/INHBA_HUMAN Inhibin beta A chain
Interferon gamma- inducible protein (IP-10) GeneSeq Accession W96709 U.S. Pat. No. 5,871,723,
P02778/CXL10_HUMAN C-X-C motif chemokine 10
Interleukin 15_vA, (IL-15_vA)
Interleukin 15_vB, (IL-15_vB)
Interleukin 15_vC, (IL-15_vC)
Interleukin 15_vD, (IL15_vD)
Interleukin 15_vE, (IL15_vE)
Interleukin 15_vF, (IL15_vF)
Interleukin- 1alpha GeneSeq Accession R45364 EP578278, P01583|IL1A_HUMAN Interleukin-1 alpha
Interleukin 2 (IL-2)
Interleukin 22, (IL22),
Interleukin 6 GeneSeq Accession R55256 JP06145063, P05231/IL6_HUMAN Interleukin-6
Interleukin 8 (IL-8) receptor GeneSeq Accession R53932 JP06100595, GenBank: AAA59159.1
Interleukin 8 receptor B GeneSeq Accession R80758 U.S. Pat. No. 5,440,021, IL-8RB,
P25025/CXCR2_HUMAN C-X-C chemokine receptor type 2
Interleukin-2 receptor associated protein p43 GeneSeq Accession R97569 WO9621732-, SEQ ID NO: 2
of WO9621732
interleukin-3 GeneSeq Accession R92801 WO9604306, P08700|IL3_HUMAN Interleukin-3
interleukin-4 muteins GeneSeq Accessions W52150, W52151, W52153, W52154, W52155, W52156,
W52157, W52158, W52159, W52160, W52161, W52162, W52163, W52164, W52165, W52166, and
W52167 WO9747744, Variants of wildtype IL-4 which has the sequence:, P05112/IL4_HUMAN
Interleukin-4, (isoform 1)
Interleukin-8 (IL-8) protein. GeneSeq Accession W96711 U.S. Pat. No. 5,871,723, P10145/IL8_HUMAN
Interleukin-8, (isoform 1)
Interleukin-9 (IL-9) mature protein (Thr117 version). GeneSeq Accession W68158 WO9827997, FIG.
2 of WO9827997
interleukin-9 GeneSeq Accession R92797 WO9604306, P15248/IL9_HUMAN Interleukin-9
Latent TGF beta binding protein II GeneSeq Accession Y70552 WO0012551,
Q14767/LTBP2_HUMAN Latent-transforming growth factor beta-binding protein 2
Leukemia Inhibitory Factor, (LIF)
Liver expressed chemokine- 1(LVEC-1) GeneSeq Accession R95689 WO9616979, SEQ ID NO: 2 of,
WO9616979
Liver expressed chemokine- 2(LVEC-2) GeneSeq Accession R95690 WO9616979, SEQ ID NO: 4 of,
WO9616979
LT-alpha GeneSeq Accession P70107 EP250000, P01374/TNFB_HUMAN Lymphotoxin-alpha
LT-beta GeneSeq Accession R56869 WO9413808, Q06643/TNFC_HUMAN Lymphotoxin-beta,
(isoform 1)
Lymphotactin GeneSeq Accession B50052 WO0073320, P47992/XCL1_HUMAN Lymphotactin
Macrophage- derived chemokine (MDC) GeneSeq Accessions W40811 and Y24414 U.S. Pat. No.
5,688,927/U.S. Pat. No. 5,932,703, O00626/CCL22_HUMAN C-C motif chemokine 22
Macrophage derived chemokine analogue MDC (n + 1) GeneSeq Accession Y24413 U.S. Pat. No.
5,932,703
Macrophage derived chemokine analogue MDC- yl GeneSeq Accession Y24415 U.S. Pat. No. 5,932,703
Macrophage derived chemokine analogue MDC-eyfy GeneSeq Accession Y24416 U.S. Pat. No. 5,932,703
(“eyfy” disclosed as SEQ ID NO: 546), Wildtype MDC is SEQ ID NO: 2 of 5,932,703
Maspin; Protease Inhibitor 5 GeneSeq Accession R50938 WO9405804, P36952/SPB5_HUMAN Serpin
B5, (isoform 1)
MCP-3 GeneSeq Accession R73915 W09509232, P80098/CCL7_HUMAN C-C motif chemokine 7
MCP-4 receptor GeneSeq Accession W56689 WO9809171, SEQ ID NO: 2 of WO9809171
MCP-I receptor GeneSeq Accession R79165 WO9519436: MCP-IA, SEQ ID NO: 2 of WO9519436,
MCP-1B, SEQ ID NO: 4 of WO9519436
MCP-Ia GeneSeq Accession R73914 WO9509232, MCP-1 provided as P13500/CCL2_HUMAN C-C
motif chemokine 2
MCP-Ib GeneSeq Accession Y26176 WO9929728, MCP-1 provided as P13500/CCL2_HUMAN C-C
motif chemokine 2
Melanoma inhibiting protein GeneSeq Accession R69811 WO9503328
Membrane bound proteins GeneSeq. Accession Y66631-Y66765 WO9963088
Membrane-Bound Transcription Factor Peptidase, Site 2, (MBTPS2)
Metl 17 human interleukin 9 GeneSeq Accession W27522 WO9708321
Microphage derived chemokine, MDC GeneSeq Accession W20058 WO9640923,
O00626/CCL22_HUMAN C-C motif chemokine 22
MIP-3 alpha GeneSeq Accession W44398 WO9801557, P78556/CCL20_HUMAN C-C motif
chemokine 20, (isoform 1)
MIP-3 beta GeneSeq Accession W44399 WO9801557, Q99731/CCL19_HUMAN C-C motif chemokine
19
MIP-Gamma GeneSeq Accession R70798, WO2006135382
Modified Rantes GeneSeq Accession W38129 WO9737005, Wildtype Rantes provided herein as
P13501/CCL5_HUMAN C-C motif chemokine 5
MP52 GeneSeq Accession W36100 WO9741250, P43026/GDF5_HUMAN Growth/differentiation
factor 5
Mutant Interleukin 6 S176R GeneSeq Accession R54990 WO9411402, S176R variant of wildtype IL-6
which has the sequence:, P05231/IL6_HUMAN Interleukin-6
myofibrillar protein troponin I GeneSeq Accession W18054 WO9719955, SEQ ID NO: 3 of WO9719955
Nerve Growth Factor-beta GeneSeq Accession R11474 EP414151, P01138/NGF_HUMAN Beta-nerve
growth factor
Nerve Growth Factor-beta2 GeneSeq Accession W69725 EP859056, FIG. 1 of EP859056
Neuropilin-1 GeneSeq Accession Y06319 WO9929858, O14786/NRP1_HUMAN Neuropilin-1,
(isoform 1)
Neuropilin-2 GeneSeq Accession Y03618 WO9929858, O60462/NRP2_HUMAN Neuropilin-2,
(isoform A22)
Neurotactin GeneSeq Accessions Y77537, W34307, Y53259, and, Y77539 U.S. Pat. No. 6,013,257
WO9742224, P78423/X3CL1_HUMAN Fractalkine
Neurotrophin- 4a GeneSeq Accession R47101 WO9325684, Wildtype neurotrophin provided as:,
P34130/NTF4_HUMAN Neurotrophin-4
Neurotrophin- 4b GeneSeq Accession R47102 WO9325684, P34130/NTF4_HUMAN Neurotrophin-4
Neurotrophin- 4c GeneSeq Accession R47103 WO9325684, P34130/NTF4_HUMAN Neurotrophin-4
Neurotrophin- 4d GeneSeq Accession R47102 WO9325684, P34130/NTF4_HUMAN Neurotrophin-4
Neurotrophin-3 GeneSeq Accession W8889 WO9821234, P20783/NTF3_HUMAN Neurotrophin-3,
(isoform 1)
Neurotrophin-4 GeneSeq Accession R47100 WO9325684, P34130/NTF4_HUMAN Neurotrophin-4
Neutrophil activating peptide-2 (NAP-2) GeneSeq Accession W96719 U.S. Pat. No. 5,871,723, SEQ ID
NO: 11 of U.S. Pat. No. 5,871,723
NOGO-66 Receptor Genbank Accession AAG53612
NOGO-A Genbank Accession CAB99248
NOGO-B Genbank Accession CAB99249
NOGO-C Genbank Accession CAB99250
Novel betachemokine designated PTEC GeneSeq Accession W27271 WO9739126, SEQ ID NO: 2 of
WO9739126
N-terminal modified chemokine GroHEK/hSDF- 1alpha GeneSeq Accession Y05820 WO9920759, SEQ
ID NO: 12 of WO9920759
N-terminal modified chemokine GroHEK/hSDF- 1beta. GeneSeq Accession Y05821 WO9920759, SEQ
ID NO: 13 of WO9920759
N-terminal modified chemokine met- hSDF-1 alpha GeneSeq Accession Y05818 WO9920759, SEQ ID
NO: 10 of WO9920759
N-terminal modified chemokine met- hSDF-1 beta GeneSeq Accession Y05819 WO9920759, SEQ ID
NO: 11 of WO9920759
OPGL GeneSeq Accession W83195 WO9846751, O14788/TNF11_HUMAN Tumor necrosis factor
ligand superfamily member 11, (isoform 1)
Osteogenic Protein-1; OP-1; BMP-7 GeneSeq Accession W34783 WO973462,
P18075/BMP7_HUMAN Bone morphogenetic protein 7
Osteogenic Protein-2 GeneSeq Accession R57973 WO9406399, P34820/BMP8B_HUMAN Bone
morphogenetic protein 8B
OX40; ACT-4 GeneSeq Accession R74737 WO9512673, P43489/TNR4_HUMAN Tumor necrosis
factor receptor superfamily member 4
OX40L GeneSeq Accession R79903 WO9521915, P23510/TNFL4_HUMAN Tumor necrosis factor
ligand superfamily member 4, (isoform 1)
Oxytocin (Neurophysin I) GeneSeq Accession B24085 and B24086 WO0053755,
P01178/NEU1_HUMAN Oxytocin-neurophysin 1
P09529/INHBB_HUMAN Inhibin beta B chain
P13497-2|BMP1_HUMAN Isoform BMP1-1 of Bone morphogenetic protein 1, (isoform BMP1-1)
P13497-3|BMP1_HUMAN Isoform BMP1-4 of Bone morphogenetic protein 1, (isoform BMP1-4)
P13497-4|BMP1_HUMAN Isoform BMP1-5 of Bone morphogenetic protein 1, (isoform BMP1-5)
P13497-5|BMP1_HUMAN Isoform BMP1-6 of Bone morphogenetic protein 1, (isoform BMP1-6)
P13497-6|BMP1_HUMAN Isoform BMP1-7 of Bone morphogenetic protein 1, (isoform BMP1-7)
P22003-2|BMP5_HUMAN Isoform 2 of Bone morphogenetic protein 5, (isoform 2)
Parathyroid hormone, P01270|PTHY_HUMAN Parathyroid hormone
Patched GeneSeq Accession W72969 U.S. Pat. No. 5,837,538, Q13635/PTC1_HUMAN Protein patched
homolog 1, (isoform L)
Patched-2 GeneSeq Accession Y43261 WO9953058, Q9Y6C5/PTC2_HUMAN Protein patched
homolog 2, (isoform 1)
PDGF-D GeneSeq Accession Y71130 WO0027879, Q9GZP0/PDGFD_HUMAN Platelet-derived
growth factor D, (isoform 1)
Pituitary expressed chemokine (PGEC) GeneSeq Accession R95691 WO9616979, SEQ ID NO: 6 of,
WO9616979
Placental Growth Factor- 2 GeneSeq Accession Y08289 DE19748734, P49763-3/PLGF_HUMAN
Isoform PIGF-2 of Placenta growth factor, (isoform PIGF-2)
Placental Growth Factor GeneSeq Accessions R23059 and R23060 WO9206194, P49763-
2/PLGF_HUMAN Isoform PIGF-1 of Placenta growth factor, (isoform PIGF-1)
Plasminogen Activator Inhibitor-2; PAI-2 Gene Seq Accession P94160 DE3722673,
P05120/PAI2_HUMAN Plasminogen activator inhibitor 2
Platelet derived growth factor Bvsis GeneSeq Accession P80595 and P80596 EP282317, FIG. 1 of
EP282317
Platelet derived growth factor GeneSeq Accession B48653 WO0066736, PDGF-A,
P04085/PDGFA_HUMAN Platelet-derived growth factor subunit A, (Isoform long), PDGF-B,
P01127/PDGFB_HUMAN Platelet-derived growth factor subunit B, (isoform 1)
Platelet derived growth factor precursor A GeneSeq Accession R84759 EP682110, PDGF-A precursor
(variant D1), PDGF-A precursor (variant 13-1)
Platelet derived growth factor precursor B GeneSeq Accession R84760 EP682110, FIG. 1 or FIG. 2,
Wildtype PDGF-B provided as:, PDGF-B, P01127/PDGFB_HUMAN Platelet-derived growth factor
subunit B, (isoform 1)
Platelet-Derived Growth Factor A chain GeneSeq Accession R38918 U.S. Pat. No. 5,219,739,
P04085/PDGFA_HUMAN Platelet-derived growth factor subunit A, (Isoform long)
Platelet-Derived Growth Factor B chain GeneSeq Accession R38919 U.S. Pat. No. 5,219,739,
P01127/PDGFB_HUMAN Platelet-derived growth factor subunit B, (isoform 1),
Preproapolipo- protein “Milano” variant 5,721,114, SEQ ID NO: 6 of U.S. Pat. No. 5,721,114
Preproapolipo- protein “Paris” variant GeneSeq Accession W08602 WO9637608
Primate CC chemokine “ILINCK” GeneSeq Accession W69990 WO98328658, SEQ ID NO: 4 from
WO9832858
Primate CXC chemokine “IBICK” GeneSeq Accession W69989 WO9832858, SEQ ID NO: 2 from
WO9832858
Prolactin GeneSeq Accession R78691 WO9521625, P01236/PRL_HUMAN Prolactin
Prolactin2 GeneSeq Accession Y31764 U.S. Pat. No. 5,955,346
Proprotein Convertase Subtilisin/Kexin Type 1, (PCSK1)
Proprotein Convertase Subtilisin/Kexin Type 2 (PCSK2)
Proprotein Convertase Subtilisin/Kexin Type 3 Sol, (PCSK3_SOL)
Proprotein Convertase Subtilisin/Kexin Type 3, (PCSK3)
Proprotein Convertase Subtilisin/Kexin Type 4, (PCSK4)
Proprotein Convertase Subtilisin/Kexin Type 5, (PCSK5)
Proprotein Convertase Subtilisin/Kexin Type 6 (PCSK6)
Proprotein Convertase Subtilisin/Kexin Type 8, (PCSK8)
Proprotein Convertase Subtilisin/Kexin Type 9, (PCSK9)
Proprotein Convertase Subtilisin/Kexin Type, (PCSK7)
Protease inhibitor peptides GeneSeq Accessions R12435, R12436, R12437, R12438, R12439, R12440,
and R1244 WO9106561
RANTES 8-68 GeneSeq Accession W29529 WO9744462, Wildtype Rantes provided herien as
P13501/CCL5_HUMAN C-C motif chemokine 5
RANTES 9-68 GeneSeq Accession W29528 WO9744462, Wildtype Rantes provided herien as
P13501/CCL5_HUMAN C-C motif chemokine 5
RANTES GeneSeq Accession Y05299 EP905240, P13501/CCL5_HUMAN C-C motif chemokine 5
RANTES peptide GeneSeq Accession W29538 WO9744462, Wildtype Rantes provided herien as
P13501/CCL5_HUMAN C-C motif chemokine 5
RANTES receptor GeneSeq Accession W29588 U.S. Pat. No. 5,652,133, SEQ ID NO: 2 of U.S. Pat. No.
5,652,133
Recombinant interleukin-16 GeneSeq Accession W33373 DE19617202, Q14005/IL16_HUMAN Pro-
interleukin-16, (isoform 1)
Resistin GeneSeq Accession W69293 WO0064920, Q9HD89/RETN_HUMAN Resistin, (isoform 1)
Retroviral protease inhibitors GeneSeq Accessions R06660, R06661, R06662, R06663, R06664,
R06665, R06666, R06667, R06668, R06669, R06670, R06671, R06672, R06673, R06674, R06675, and
R06676 EP387231
Secreted and Transmembrane polypeptides GeneSeq Accession B44241-B44334 WO0053756
Secreted and Transmembrane polypeptides GeneSeq Accession Y41685-Y41774 WO9946281
Serpin Family A Member 1, (SERPINA1)
Serpin Peptidase Inhibitor, Clade B (Ovalbumin), Member 1, (SERPINB1)
Soluble BMP Receptor Kinase Protein- 3 GeneSeq Accession R95227 WO9614579,
Q13873/BMPR2_HUMAN Bone morphogenetic protein receptor type-2
Soluble VEGF Receptor GeneSeq Accession W47037 U.S. Pat. No. 5,712,380; sVEGF-RI (FIG. 3) of
U.S. Pat. No. 5,712,380, sVEGF-RII (FIG. 11) of U.S. Pat. No. 5,712,380, sVEGF-RTMI (FIG. 15) of
U.S. Pat. No. 5,712,380, sVEGF-RTMII (FIG. 13) of U.S. Pat. No. 5,712,380
Stem Cell Inhibitory Factor GeneSeq Accession R11553 WO9104274, SCIF in Table I of WO9104274,
SCIF in Table II of WO9104274
Stromal Derived Factor- 1 alpha GeneSeq Accession Y39995 WO9948528, P48061-2/SDF1_HUMAN
Isoform Alpha of Stromal cell-derived factor 1, (isoform alpha)
Stromal Derived Factor- 1 beta GeneSeq Accession R75420 CA2117953, P48061/SDF1_HUMAN
Stromal cell-derived factor 1, (isoform beta)
Substance P (tachykinin) GeneSeq Accession B23027 WO0054053
TACI GeneSeq Accession W75783 WO9839361, O14836/TR13B_HUMAN Tumor necrosis factor
receptor superfamily member 13B, (isoform 1)
Tarc GeneSeq Accession W14917 WO9711969, Q92583/CCL17_HUMAN C-C motif chemokine 17
TGF-beta 1 GeneSeq Accession R29657 WO9216228, P01137/TGFB1_HUMAN Transforming growth
factor beta-1
TGF-beta 2 GeneSeq Accession R39659 EP542679, P61812/TGFB2_HUMAN Transforming growth
factor beta-2
Thrl 17 human interleukin 9 GeneSeq Accession W27521 WO9708321, P15248|IL9_HUMAN
Interleukin-9
Thrombopoietin derivative 3 GeneSeq Accession Y77262, WO0000612 (e.g. Table 3), Wildtype
thrombopoietin provided as:, P40225|TPO_HUMAN Thrombopoietin, (isoform 1)
Thrombopoietin derivative 4 GeneSeq Accession Y77267, WO0000612 (e.g. Table 3), Wildtype
thrombopoietin provided as:, P40225|TPO_HUMAN Thrombopoietin, (isoform 1)
Thrombopoietin derivative 5 GeneSeq Accession Y77246, WO0000612 (e.g. Table 3), Wildtype
thrombopoietin provided as:, P40225|TPO_HUMAN Thrombopoietin, (isoform 1)
Thrombopoietin derivative 6 GeneSeq Accession Y77253, WO0000612 (e.g. Table 3), Wildtype
thrombopoietin provided as:, P40225|TPO_HUMAN Thrombopoietin, (isoform 1)
Thrombopoietin derivative, 7 GeneSeq Accession Y77256, WO0000612 (e.g. Table 3), Wildtype
thrombopoietin provided as:, P40225|TPO_HUMAN Thrombopoietin, (isoform 1)
Thrombopoietin derivative1 GeneSeq Accession Y77244 WO0000612 (e.g. Table 3), Wildtype
thrombopoietin provided as:, P40225|TPO_HUMAN Thrombopoietin, (isoform 1)
Thrombopoietin derivative2 GeneSeq Accession Y77255 WO0000612 (e.g. Table 3), Wildtype
thrombopoietin provided as:, P40225|TPO_HUMAN Thrombopoietin, (isoform 1)
Thrombopoietin GeneSeq Accession R79905 WO9521920, P40225|TPO_HUMAN Thrombopoietin,
(isoform 1)
Thymus expressed chemokine (TECK) GeneSeq Accession W44397 WO9801557,
O15444/CCL25_HUMAN C-C motif chemokine 25
Tim-1 protein GeneSeq Accession Y28290 WO9933990, SEQ ID NO: 2 of, WO9933990
TNF-alpha precursor GeneSeq Accession P60074 EP205038
TNF-R GeneSeq Accession R10986 AU9058976, P19438/TNR1A_HUMAN Tumor necrosis factor
receptor superfamily member 1A, (isoform 1)
TNF-RII; TNF p75 receptor; Death Receptor GeneSeq Accession R11141 EP418014,
P20333/TNR1B_HUMAN Tumor necrosis factor receptor superfamily member 1B, (isoform 1)
Transthyretin (TTR), Gene ID: 7276
Troponin peptides GeneSeq Accessions Y29581, Y29582, Y29583, Y29584, Y29585, and Y29586
WO9933874. Wildtype troponins provided as: Human fast twitch skeletal muscle troponin C GeneSeq
Accession W22597 W09730085, P02585/TNNC2_HUMAN Troponin C, skeletal muscle, Human fast
twitch skeletal muscle troponin I GeneSeq Accession W18054 W09730085, P48788/TNNI2_HUMAN
Troponin I, fast skeletal muscle, (isoform 1), Human fast twitch skeletal muscle troponin T GeneSeq
Accession W22599 W09730085, SEQ ID NO: 3 of WO9730085, fragment. myofibrillar protein troponin
I GeneSeq Accession W18053 W09719955, SEQ ID NO: 3 of WO9719955, Human fast twitch skeletal
muscle Troponin subunit C GeneSeq Accession B00134 WO0054770, SEQ ID NO: 1 of WO0054770,
Human fast twitch skeletal muscle Troponin subunit I Protein GeneSeq Accession B00135 WO0054770,
SEQ ID NO: 2 of WO0054770, Human fast twitch skeletal muscle Troponin subunit T GeneSeq
Accession B00136 WO0054770, SEQ ID NO: 3 of WO0054770,
Truncated monocyte chemotactic protein 2 (6-76) GeneSeq Accession Y07234 EP906954, FIG. 1 of
EP905241 and EP906954
Truncated monocyte chemotactic protein 2 (6-76) GeneSeq Accession Y07238 EP905241, FIG. 1 of
EP905241 and EP906954
Truncated RANTES protein (3-68) GeneSeq Accessions Y07236 and Y07232 EP905241; EP906954,
FIG. 1 of EP906954
Vasopressin (Neurophysin II) GeneSeq Accession B24085 and B24086 WO0053755,
P01185/NEU2_HUMAN Vasopressin-neurophysin 2-copeptin
VEGF R-3; flt-4 GeneSeq Accession B29047 WO0058511, P35916/VGFR3_HUMAN Vascular
endothelial growth factor receptor 3, (isoform 1)
VEGF Receptor; KDR; flk-1 GeneSeq Accession W69679 WO9831794, P35968/VGFR2_HUMAN
Vascular endothelial growth factor receptor 2, (isoform 1)
VEGF-110 GeneSeq Accession Y69417 WO0013702, SEQ ID NO: 11 from WO0013702
VEGF-121 GeneSeq Accession B50432 WO0071713, SEQ ID NO: 2 from WO0071713
VEGF-138 GeneSeq Accession Y43483 WO9940197, SEQ ID NO: 4 of WO99/40197
VEGF-145 GeneSeq Accession Y69413 WO0013702, SEQ ID NO: 4 from WO0013702
VEGF-162 GeneSeq Accession Y43484 W09940197, SEQ ID NO: 8 of WO99/40197
VEGF-165 GeneSeq Accession Y69414 WO0013702, SEQ ID NO: 6 from WO0013702
VEGF-182 GeneSeq Accession Y43483 W09940197, SEQ ID NO: 6 of WO99/40197
VEGF-189 GeneSeq Accession Y69415 WO0013702, SEQ ID NO: 8 from WO0013702
VEGF-206 GeneSeq Accession Y69416 W00013702, SEQ ID NO: 10 from WO0013702
VEGF-D GeneSeq Accession W53240 WO9807832, O43915/VEGFD_HUMAN Vascular endothelial
growth factor D
VEGF-E; VEGF-X GeneSeq Accession Y33679 WO9947677, SEQ ID NO: 2 from WO9947677
Wild type monocyte chemotactic protein 2 GeneSeq Accession Y07233 EP906954,
P80075/CCL8_HUMAN C-C motif chemokine 8
Wild type monocyte chemotactic protein 2 GeneSeq Accession Y07237 EP905241,
P80075/CCL8_HUMAN C-C motif chemokine 8
ZTGF-beta 9 GeneSeq Accession Y70654 WO0015798, SEQ ID NO: 2 of WO0015798

TABLE 3
Illustrative indications and related genes

| Category | Disease | Genes | Entrez ID |
|---|---|---|---|
| Disorders of | Galactosemia | GALT, GALK1, GALE | 2592, 2584, 2582 |
| carbohydrate | Essential fructosuria | KHK | 3795 |
| metabolism | Hereditary fructose | ALDOB | 229 |
| | intolerance | | |
| | Glycogen storage disease | G6PC, SLC37A4, SLC17A3 | 2538, 2542, 10786 |
| | type I | | |
| | Glycogen storage disease | GAA | 2548 |
| | type II | | |
| | Glycogen storage disease | AGL | 178 |
| | type III | | |
| | Glycogen storage disease | GBE1 | 2632 |
| | type IV | | |
| | Glycogen storage disease | PYGM | 5837 |
| | type V | | |
| | Glycogen storage disease | PYGL | 5836 |
| | type VI | | |
| | Glycogen storage disease | PYGM | 5837 |
| | type VII | | |
| | Glycogen storage disease | PHKA1, PHKA2, PHKB, | 5255, 5256, 5257, |
| | type IX | PHKG1, PHKG2 | 5260, 5261 |
| | Glycogen storage disease | SLC2A2 | 6514 |
| | type XI | | |
| | Glycogen storage disease | ALDOA | 226 |
| | type XII | | |
| | Glycogen storage disease | ENO1, ENO2, ENO3 | 2023, 2026, 2027 |
| | type XIII | | |
| | Glycogen storage disease | GYS1, GYS2 | 2997, 2998 |
| | type 0 | | |
| | Pyruvate carboxylase | PC | 5091 |
| | deficiency | | |
| | Pyruvate kinase deficiency | PKLR | 5313 |
| | Transaldolase deficiency | TALDO1 | 6888 |
| | Triosephosphate isomerase | TPI1 | 7167 |
| | deficiency | | |
| | Fructose bisphosphatase | FBP1 | 2203 |
| | deficiency | | |
| | Hyperoxaluria | AGXT, GRHPR | 189, 9380 |
| | Hexokinase deficiency | HK1 | 3098 |
| | Glucose-galactose | SLC5A1 | 6523 |
| | malabsorption | | |
| | Glucose-6-phosphate | G6PD | 2539 |
| | dehydrogenase deficiency | | |
| Disorders of | Alkaptonuria | HGD | 3081 |
| amino acid | Aspartylglucosaminuria | AGA | 175 |
| metabolism | Methylmalonic acidemia | MUT, MCEE, MMAA, MMAB, | 4594, 84693, 166785, |
| | | MMACHC, MMADHC, LMBRD1 | 326625, 25974, 27249, |
| | | | 55788 |
| | Maple syrup urine disease | BCKDHA, BCKDHB, | 593, 594, 1629, 1738 |
| | | DBT, DLD | |
| | Homocystinuria | CBS | 875 |
| | Tyrosinemia | FAH, TAT, HPD | 2184, 6898, 3242 |
| | Trimethylaminuria | FMO3 | 2328 |
| | Hartnup disease | SLC6A19 | 340024 |
| | Biotinidase deficiency | BTD | 686 |
| | Ornithine | OTC | 5009 |
| | carbamoyltransferase | | |
| | deficiency | | |
| | Carbamoyl-phosphate | CPS1 | 1373 |
| | synthase I deficiency disease | | |
| | Citrullinemia | ASS, SLC25A13 | 445, 10165 |
| | Hyperargininemia | ARG1 | 383 |
| | Hyperhomocysteinemia | MTHFR | 4524 |
| | Hypermethioninemia | MAT1A, GNMT, | 4143, 27232, 191 |
| | | AHCY | |
| | Hyperlysinemias | AASS | 10157 |
| | Nonketotic hyperglycinemia | GLDC, AMT, GCSH | 2731, 275, 2653 |
| | Propionic acidemia | PCCA, PCCB | 5095, 5096 |
| | Hyperprolinemia | ALDH4A1, PRODH | 8659, 5625 |
| | Cystinuria | SLC3A1, SLC7A9 | 6519, 11136 |
| | Dicarboxylic aminoaciduria | SLC1A1 | 6505 |
| | Glutaric acidemia type 2 | ETFA, ETFB, ETFDH | 2108, 2109, 2110 |
| | Isovaleric acidemia | IVD | 3712 |
| | 2-Hydroxyglutaric aciduria | L2HGDH, D2HGDH | 79944, 728294 |
| Disorders of | N-Acetylglutamate synthase | NAGS | 162417 |
| the urea cycle | deficiency | | |
| | Argininosuccinic aciduria | ASL | 435 |
| | Argininemia | ARG1 | 383 |
| Disorders of | Very long-chain acyl- | ACADVL | 37 |
| fatty acid | coenzyme A dehydrogenase | | |
| metabolism | deficiency | | |
| | Long-chain 3-hydroxyacyl- | HADHA | 3030 |
| | coenzyme A dehydrogenase | | |
| | deficiency | | |
| | Medium-chain acyl- | ACADM | 34 |
| | coenzyme A dehydrogenase | | |
| | deficiency | | |
| | Short-chain acyl-coenzyme A | ACADS | 35 |
| | dehydrogenase deficiency | | |
| | 3-hydroxyacyl-coenzyme A | HADH | 3033 |
| | dehydrogenase deficiency | | |
| | 2,4 Dienoyl-CoA reductase | NADK2 | 133686 |
| | deficiency | | |
| | 3-Hydroxy-3-methylglutaryl- | HMGCL | 3155 |
| | CoA lyase deficiency | | |
| | Malonyl-CoA decarboxylase | MLYCD | 23417 |
| | deficiency | | |
| | Systemic primary carnitine | SLC22A5 | 6584 |
| | deficiency | | |
| | Carnitine-acylcarnitine | SLC25A20 | 788 |
| | translocase deficiency | | |
| | Carnitine | CPT1A | 1374 |
| | palmitoyltransferase I | | |
| | deficiency | | |
| | Carnitine | CPT2 | 1376 |
| | palmitoyltransferase II | | |
| | deficiency | | |
| | Lysosomal acid lipase | LIPA | 3988 |
| | deficiency | | |
| | Gaucher's disease | GBA | 2629 |
| Disorders of | Acute intermittent porphyria | HMBS | 3145 |
| porphyrin | Gunther disease | UROS | 7390 |
| metabolism | Porphyria cutanea tarda | UROD | 7389 |
| | Hepatoerythropoietic | UROD | 7389 |
| | porphyria | | |
| | Hereditary coproporphyria | CPOX | 1371 |
| | Variegate porphyria | PPOX | 5498 |
| | Erythropoietic protoporphyria | FECH | 2235 |
| | Aminolevulinic acid | ALAD | 210 |
| | dehydratase deficiency | | |
| | porphyria | | |
| Lysosomal | Farber disease | ASAH1 | 427 |
| storage | Krabbe disease | GALC | 2581 |
| disorders | Galactosialidosis | CTSA | 5476 |
| | Fabry disease | GLA | 2717 |
| | Schindler disease | NAGA | 4668 |
| | GM1 gangliosidosis | GLB1 | 2720 |
| | Tay-Sachs disease | HEXA | 3073 |
| | Sandhoff disease | HEXB | 3074 |
| | GM2-gangliosidosis, AB | GM2A | 2760 |
| | variant | | |
| | Niemann-Pick disease | SMPD1, NPC1, NPC2 | 6609, 4864, 10577 |
| | Metachromatic | ARSA, PSAP | 410, 5660 |
| | leukodystrophy | | |
| | Multiple sulfatase deficiency | SUMF1 | 285362 |
| | Hurler syndrome | IDUA | 3425 |
| | Hunter syndrome | IDS | 3423 |
| | Sanfilippo syndrome | SGSH, NAGLU, | 6448, 4669, 138050, |
| | | HGSNAT, GNS | 2799 |
| | Morquio syndrome | GALNS, GLB1 | 2588, 2720 |
| | Maroteaux-Lamy syndrome | ARSB | 411 |
| | Sly syndrome | GUSB | 2990 |
| | Sialidosis | NEU1, NEU2, NEU3, | 4758, 4759, 10825, |
| | | NEU4 | 129807 |
| | I-cell disease | GNPTAB, GNPTG | 79158, 84572 |
| | Mucolipidosis type IV | MCOLN1 | 57192 |
| | Infantile neuronal ceroid | PPT1, PPT2 | 5538, 9374 |
| | lipofuscinosis | | |
| | Jansky-Bielschowsky disease | TPP1 | 1200 |
| | Batten disease | CLN1, CLN2, CLN3, | 5538, 1200, 1201, 1203, |
| | | CLN5, CLN6, MFSD8, | 54982, 256471, 2055, 1509 |
| | | CLN8, CTSD | |
| | Kufs disease, Type A | CLN6, PPT1 | 54982, 5538 |
| | Kufs disease, Type B | DNAJC5, CTSF | 80331, 8722 |
| | Alpha-mannosidosis | MAN2B1, MAN2B2, | 4125, 23324, 4123 |
| | | MAN2C1 | |
| | Beta-mannosidosis | MANBA | 4226 |
| | Fucosidosis | FUCA1 | 2517 |
| | Cystinosis | CTNS | 1497 |
| | Pycnodysostosis | CTSK | 1513 |
| | Salla disease | SLC17A5 | 26503 |
| | Infantile free sialic acid | SLC17A5 | 26503 |
| | storage disease | | |
| | Danon disease | LAMP2 | 3920 |
| Peroxisome | Zellweger syndrome | PEX1, PEX2, PEX3, | 5189, 5828, 8504, 5830, |
| biogenesis | | PEX5, PEX6, PEX12, | 5190, 5193, 5195, 55670 |
| disorders | | PEX14, PEX26 | |
| | Infantile Refsum disease | PEX1, PEX2, PEX26 | 5189, 5828, 55670 |
| | Neonatal | PEX5, PEX1, PEX10, | 5830, 5189, 5192, 5194, |
| | adrenoleukodystrophy | PEX13, PEX26 | 55670 |
| | RCDP Type 1 | PEX7 | 5191 |
| | Pipecolic acidemia | PAHX | 5264 |
| | Acatalasia | CAT | 847 |
| | Hyperoxaluria type 1 | AGXT | 189 |
| | Acyl-CoA oxidase deficiency | ACOX1 | 51 |
| | D-bifunctional protein | HSD17B4 | 3295 |
| | deficiency | | |
| | Dihydroxyacetonephosphate | GNPAT | 8443 |
| | acyltransferase deficiency | | |
| | X-linked | ABCD1 | 215 |
| | adrenoleukodystrophy | | |
| | α-Methylacyl-CoA racemase | AMACR | 23600 |
| | deficiency | | |
| | RCDP Type 2 | DHAPAT | 8443 |
| | RCDP Type 3 | AGPS | 8540 |
| | Adult Refsum disease-1 | PHYH | 5264 |
| | Mulibrey nanism | TRIM37 | 4591 |
| Disorders of | Lesch-Nyhan syndrome | HPRT | 3251 |
| purine or | Adenine | APRT | 353 |
| pyrimidine | phosphoribosyltransferase | | |
| metabolism | deficiency | | |
| | Adenosine deaminase | ADA | 100 |
| | deficiency | | |
| | Adenosine monophosphate | AMPD1 | 270 |
| | deaminase deficiency type 1 | | |
| | Adenylosuccinate lyase | ADSL | 158 |
| | deficiency | | |
| | Dihydropyrimidine | DPYD | 1806 |
| | dehydrogenase deficiency | | |
| | Miller syndrome | DHODH | 1723 |
| | Orotic aciduria | UMPS | 7372 |
| | Purine nucleoside | PNP | 4860 |
| | phosphorylase deficiency | | |
| | Xanthinuria | XDH, MOCS1, | 7498, 4337, 4338, 10243 |
| | | MOCS2, GEPH | |

The Entrez entries listed in the table above are hereby incorporated by reference in their entireties.

For each of the illustrative proteins, peptides, or genes identified in Table 1, Table 2, or Table 3, the present disclosure considers and covers natural or engineered variants, family members, orthologues, fragments or fusions construct thereof of the illustrative proteins, peptides, or genes.

### Definitions

The following definitions are used in connection with the invention disclosed herein. Unless defined otherwise, all technical and scientific terms used herein have the same meaning as commonly understood to one of skill in the art to which this invention belongs.

The term “in vivo” refers to an event that takes place in a subject's body.

The term “ex vivo” refers to an event which involves treating or performing a procedure on a cell, tissue and/or organ which has been removed from a subject's body. Aptly, the cell, tissue and/or organ may be returned to the subject's body in a method of treatment or surgery.

As used herein, the term “variant” encompasses but is not limited to nucleic acids or proteins which comprise a nucleic acid or amino acid sequence which differs from the nucleic acid or amino acid sequence of a reference by way of one or more substitutions, deletions and/or additions at certain positions. The variant may comprise one or more conservative substitutions. Conservative substitutions may involve, e.g., the substitution of similarly charged or uncharged amino acids.

“Carrier” or “vehicle” as used herein refer to carrier materials suitable for drug administration. Carriers and vehicles useful herein include any such materials known in the art, e.g., any liquid, gel, solvent, liquid diluent, solubilizer, surfactant, lipid or the like, which is nontoxic and which does not interact with other components of the composition in a deleterious manner.

The phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

The terms “pharmaceutically acceptable carrier” or “pharmaceutically acceptable excipient” are intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the invention is contemplated. Additional active pharmaceutical ingredients, such as other drugs, can also be incorporated into the described compositions and methods.

As used herein, “a,” “an,” or “the” can mean one or more than one.

Herein the term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 15%, up to 10%, up to 5%, or up to 1% of a given value. Further, the term “about” when used in connection with a referenced numeric indication means the referenced numeric indication plus or minus up to 10% of that referenced numeric indication. For example, the language “about 50” covers the range of 45 to 55. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

The terms “comprise”, “comprising”, “contain,” “containing,” “including”, “includes”, “having”, “has”, “with”, or variants thereof as used in either the present disclosure and/or in the claims, are intended to be inclusive in a manner similar to the term “comprising.” Although the open-ended term “comprising,” is used herein to describe and claim the invention, the present invention, or embodiments thereof, may alternatively be described using alternative terms such as “consisting of” or “consisting essentially of.”

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

By treating is meant, at least, ameliorating or avoiding the effects of a disease, including reducing a sign or symptom of the disease.

Any aspect or embodiment described herein can be combined with any other aspect or embodiment as disclosed herein.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features herein set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

### SEQUENCES

In embodiments, the present disclosure provides for any of the sequences below, or a sequence having at least about 95%, or at least about 97%, or at least about 98% identity thereto, e.g. as compositions and/or for use in the methods described herein.

In embodiments, the GFP part of a sequence (see the bold and underlined text) may be swapped with a different coding sequence to yield a circRNA.

>looseHairpin_GFP
AAGCTTTAATACGACTCACTATAGGGTTTAAAAAAGCCACCATGGTGAGCAAGGGCGAGGAGCTGTTC
ACCGGGGTGGTGCCCATCCTGGTCGAGCTGGACGGCGACGTAAACGGCCACAAGTTCAGCGTGTCCGG
CGAGGGCGAGGGCGATGCCACCTACGGCAAGCTGACCCTGAAGTTCATCTGCACCACCGGCAAGCTGC
CCGTGCCCTGGCCCACCCTCGTGACCACCCTGACCTACGGCGTGCAGTGCTTCAGCCGCTACCCCGAC
CACATGAAGCAGCACGACTTCTTCAAGTCCGCCATGCCCGAAGGCTACGTCCAGGAGCGCACCATCTT
CTTCAAGGACGACGGCAACTACAAGACCCGCGCCGAGGTGAAGTTCGAGGGCGACACCCTGGTGAACC
GCATCGAGCTGAAGGGCATCGACTTCAAGGAGGACGGCAACATCCTGGGGCACAAGCTGGAGTACAAC
TACAACAGCCACAACGTCTATATCATGGCCGACAAGCAGAAGAACGGCATCAAGGTGAACTTCAAGAT
CCGCCACAACATCGAGGACGGCAGCGTGCAGCTCGCCGACCACTACCAGCAGAACACCCCCATCGGCG
ACGGCCCCGTGCTGCTGCCCGACAACCACTACCTGAGCACCCAGTCCGCCCTGAGCAAAGACCCCAAC
GAGAAGCGCGATCACATGGTCCTGCTGGAGTTCGTGACCGCCGCCGGGATCACTCTCGGCATGGACGA
GCTGTACAAGTACTCAGATCTCGAGCTCAAGTAGataaataaaaacccaaaccctgacgggtttaaaa
gaagagcgaattc
>tightHairpin_GFP
AAGCTTTAATACGACTCACTATAGGGTCTAAAAAAgccaccatggtgagcaagAAAAAAGCCACCATG
GTGAGCAAGGGCGAGGAGCTGTTCACCGGGGTGGTGCCCATCCTGGTCGAGCTGGACGGCGACGTAAA
CGGCCACAAGTTCAGCGTGTCCGGCGAGGGCGAGGGCGATGCCACCTACGGCAAGCTGACCCTGAAGT
TCATCTGCACCACCGGCAAGCTGCCCGTGCCCTGGCCCACCCTCGTGACCACCCTGACCTACGGCGTG
CAGTGCTTCAGCCGCTACCCCGACCACATGAAGCAGCACGACTTCTTCAAGTCCGCCATGCCCGAAGG
CTACGTCCAGGAGCGCACCATCTTCTTCAAGGACGACGGCAACTACAAGACCCGCGCCGAGGTGAAGT
TCGAGGGCGACACCCTGGTGAACCGCATCGAGCTGAAGGGCATCGACTTCAAGGAGGACGGCAACATC
CTGGGGCACAAGCTGGAGTACAACTACAACAGCCACAACGTCTATATCATGGCCGACAAGCAGAAGAA
CGGCATCAAGGTGAACTTCAAGATCCGCCACAACATCGAGGACGGCAGCGTGCAGCTCGCCGACCACT
ACCAGCAGAACACCCCCATCGGCGACGGCCCCGTGCTGCTGCCCGACAACCACTACCTGAGCACCCAG
TCCGCCCTGAGCAAAGACCCCAACGAGAAGCGCGATCACATGGTCCTGCTGGAGTTCGTGACCGCCGC
CGGGATCACTCTCGGCATGGACGAGCTGTACAAGTACTCAGATCTCGAGCTCAAGTAGCAAGTACTCA
GATCTCGAGCTCAAGTAGataaataaagaccccccagatgactctgggaattc
>free/bound GFP
AAGCTTTAATACGACTCACTATAGGGCCGAGCGAAGCGTAAAAAAGCCACCATGGTGAGCAAGGGCGA
GGAGCTGTTCACCGGGGTGGTGCCCATCCTGGTCGAGCTGGACGGCGACGTAAACGGCCACAAGTTCA
GCGTGTCCGGCGAGGGCGAGGGCGATGCCACCTACGGCAAGCTGACCCTGAAGTTCATCTGCACCACC
GGCAAGCTGCCCGTGCCCTGGCCCACCCTCGTGACCACCCTGACCTACGGCGTGCAGTGCTTCAGCCG
CTACCCCGACCACATGAAGCAGCACGACTTCTTCAAGTCCGCCATGCCCGAAGGCTACGTCCAGGAGC
GCACCATCTTCTTCAAGGACGACGGCAACTACAAGACCCGCGCCGAGGTGAAGTTCGAGGGCGACACC
CTGGTGAACCGCATCGAGCTGAAGGGCATCGACTTCAAGGAGGACGGCAACATCCTGGGGCACAAGCT
GGAGTACAACTACAACAGCCACAACGTCTATATCATGGCCGACAAGCAGAAGAACGGCATCAAGGTGA
ACTTCAAGATCCGCCACAACATCGAGGACGGCAGCGTGCAGCTCGCCGACCACTACCAGCAGAACACC
CCCATCGGCGACGGCCCCGTGCTGCTGCCCGACAACCACTACCTGAGCACCCAGTCCGCCCTGAGCAA
AGACCCCAACGAGAAGCGCGATCACATGGTCCTGCTGGAGTTCGTGACCGCCGCCGGGATCACTCTCG
GCATGGACGAGCTGTACAAGTACTCAGATCTCGAGCTCAAGTAGACGCTTCGCTCAAATTAAAAGAAG
AGCGAATTC
>crPV_GFP
AAGCTTTAATACGACTCACTATAGGGCCGAGCGAAGCGTAAAGAAAAGAAAAAGAAAAAAGAAAGCAA
AAATGTGATCTTGCTTGTAAATACAATTTTGAGAGGTTAATAAATTACAAGTAGTGCTATTTTTGTAT
TTAGGTTAGCTATTTAGCTTTACGTTCCAGGATGCCTAGTGGCAGCCCCACAATATCCAGGAAGCCCT
CTCTGCGGTTTTTCAGATTAGGTAGTCGAAAAACCTAAGAAATTTAATGGTGAGCAAGGGCGAGGAGC
TGTTCACCGGGGTGGTGCCCATCCTGGTCGAGCTGGACGGCGACGTAAACGGCCACAAGTTCAGCGTG
TCCGGCGAGGGCGAGGGCGATGCCACCTACGGCAAGCTGACCCTGAAGTTCATCTGCACCACCGGCAA
GCTGCCCGTGCCCTGGCCCACCCTCGTGACCACCCTGACCTACGGCGTGCAGTGCTTCAGCCGCTACC
CCGACCACATGAAGCAGCACGACTTCTTCAAGTCCGCCATGCCCGAAGGCTACGTCCAGGAGCGCACC
ATCTTCTTCAAGGACGACGGCAACTACAAGACCCGCGCCGAGGTGAAGTTCGAGGGCGACACCCTGGT
GAACCGCATCGAGCTGAAGGGCATCGACTTCAAGGAGGACGGCAACATCCTGGGGCACAAGCTGGAGT
ACAACTACAACAGCCACAACGTCTATATCATGGCCGACAAGCAGAAGAACGGCATCAAGGTGAACTTC
AAGATCCGCCACAACATCGAGGACGGCAGCGTGCAGCTCGCCGACCACTACCAGCAGAACACCCCCAT
CGGCGACGGCCCCGTGCTGCTGCCCGACAACCACTACCTGAGCACCCAGTCCGCCCTGAGCAAAGACC
CCAACGAGAAGCGCGATCACATGGTCCTGCTGGAGTTCGTGACCGCCGCCGGGATCACTCTCGGCATG
GACGAGCTGTACAAGTACTCAGATCTCGAGCTCAAGTAGAAAGAAAAGAAAAAGAAAAAAGACGCTTC
GCTCAAATTAAAAGAAGAGCGAATTC
>EMCV_GFP
AAGCTTTAATACGACTCACTATAGGGCCGAGCGAAGCGTAAAGAAAAGAAAAAGAAAAAAGCCCCTCT
CCCTCCCCCCCCCCTAACGTTACTGGCCGAAGCCGCTTGGAATAAGGCCGGTGTGCGTTTGTCTATAT
GTTATTTTCCACCATATTGCCGTCTTTTGGCAATGTGAGGGCCCGGAAACCTGGCCCTGTCTTCTTGA
CGAGCATTCCTAGGGGTCTTTCCCCTCTCGCCAAAGGAATGCAAGGTCTGTTGAATGTCGTGAAGGAA
GCAGTTCCTCTGGAAGCTTCTTGAAGACAAACAACGTCTGTAGCGACCCTTTGCAGGCAGCGGAACCC
CCCACCTGGCGACAGGTGCCTCTGCGGCCAAAAGCCACGTGTATAAGATACACCTGCAAAGGCGGCAC
AACCCCAGTGCCACGTTGTGAGTTGGATAGTTGTGGAAAGAGTCAAATGGCTCTCCTCAAGCGTATTC
AACAAGGGGCTGAAGGATGCCCAGAAGGTACCCCATTGTATGGGATCTGATCTGGGGCCTCGGTGCAC
ATGCTTTACATGTGTTTAGTCGAGGTTAAAAAACGTCTAGGCCCCCCGAACCACGGGGACGTGGTTTT
CCTTTGAAAAACACGATGATAATATGGCCACAACCATGATGGTGAGCAAGGGCGAGGAGCTGTTCACC
GGGGTGGTGCCCATCCTGGTCGAGCTGGACGGCGACGTAAACGGCCACAAGTTCAGCGTGTCCGGCGA
GGGCGAGGGCGATGCCACCTACGGCAAGCTGACCCTGAAGTTCATCTGCACCACCGGCAAGCTGCCCG
TGCCCTGGCCCACCCTCGTGACCACCCTGACCTACGGCGTGCAGTGCTTCAGCCGCTACCCCGACCAC
ATGAAGCAGCACGACTTCTTCAAGTCCGCCATGCCCGAAGGCTACGTCCAGGAGCGCACCATCTTCTT
CAAGGACGACGGCAACTACAAGACCCGCGCCGAGGTGAAGTTCGAGGGCGACACCCTGGTGAACCGCA
TCGAGCTGAAGGGCATCGACTTCAAGGAGGACGGCAACATCCTGGGGCACAAGCTGGAGTACAACTAC
AACAGCCACAACGTCTATATCATGGCCGACAAGCAGAAGAACGGCATCAAGGTGAACTTCAAGATCCG
CCACAACATCGAGGACGGCAGCGTGCAGCTCGCCGACCACTACCAGCAGAACACCCCCATCGGCGACG
GCCCCGTGCTGCTGCCCGACAACCACTACCTGAGCACCCAGTCCGCCCTGAGCAAAGACCCCAACGAG
AAGCGCGATCACATGGTCCTGCTGGAGTTCGTGACCGCCGCCGGGATCACTCTCGGCATGGACGAGCT
GTACAAGTACTCAGATCTCGAGCTCAAGTAGAAAGAAAAGAAAAAGAAAAAAGACGCTTCGCTCAAAT
TAAAAGAAGAGCGAATTC
>CVB_GFP
AAGCTTTAATACGACTCACTATAGGGCCGAGCGAAGCGTAAAAAATTAAAACAGCCTGTGGGTTGATC
CCACCCACAGGCCCATTGGGCGCTAGCACTCTGGTATCACGGTACCTTTGTGCGCCTGTTTTATACCC
CCTCCCCCAACTGTAACTTAGAAGTAACACACACCGATCAACAGTCAGCGTGGCACACCAGCCACGTT
TTGATCAAGCACTTCTGTTACCCCGGACTGAGTATCAATAGACTGCTCACGCGGTTGAAGGAGAAAGC
GTTCGTTATCCGGCCAACTACTTCGAAAAACCTAGTAACACCGTGGAAGTTGCAGAGTGTTTCGCTCA
GCACTACCCCAGTGTAGATCAGGTCGATGAGTCACCGCATTCCCCACGGGCGACCGTGGCGGTGGCTG
CGTTGGCGGCCTGCCCATGGGGAAACCCATGGGACGCTCTAATACAGACATGGTGCGAAGAGTCTATT
GAGCTAGTTGGTAGTCCTCCGGCCCCTGAATGCGGCTAATCCTAACTGCGGAGCACACACCCTCAAGC
CAGAGGGCAGTGTGTCGTAACGGGCAACTCTGCAGCGGAACCGACTACTTTGGGTGTCCGTGTTTCAT
TTTATTCCTATACTGGCTGCTTATGGTGACAATTGAGAGATCGTTACCATATAGCTATTGGATTGGCC
ATCCGGTGACTAATAGAGCTATTATATATCCCTTTGTTGGGTTTATACCACTTAGCTTGAAAGAGGTT
AAAACATTACAATTCATTGTTAAGTTGAATACAGCAAAATGGTGAGCAAGGGCGAGGAGCTGTTCACC
GGGGTGGTGCCCATCCTGGTCGAGCTGGACGGCGACGTAAACGGCCACAAGTTCAGCGTGTCCGGCGA
GGGCGAGGGCGATGCCACCTACGGCAAGCTGACCCTGAAGTTCATCTGCACCACCGGCAAGCTGCCCG
TGCCCTGGCCCACCCTCGTGACCACCCTGACCTACGGCGTGCAGTGCTTCAGCCGCTACCCCGACCAC
ATGAAGCAGCACGACTTCTTCAAGTCCGCCATGCCCGAAGGCTACGTCCAGGAGCGCACCATCTTCTT
CAAGGACGACGGCAACTACAAGACCCGCGCCGAGGTGAAGTTCGAGGGCGACACCCTGGTGAACCGCA
TCGAGCTGAAGGGCATCGACTTCAAGGAGGACGGCAACATCCTGGGGCACAAGCTGGAGTACAACTAC
AACAGCCACAACGTCTATATCATGGCCGACAAGCAGAAGAACGGCATCAAGGTGAACTTCAAGATCCG
CCACAACATCGAGGACGGCAGCGTGCAGCTCGCCGACCACTACCAGCAGAACACCCCCATCGGCGACG
GCCCCGTGCTGCTGCCCGACAACCACTACCTGAGCACCCAGTCCGCCCTGAGCAAAGACCCCAACGAG
AAGCGCGATCACATGGTCCTGCTGGAGTTCGTGACCGCCGCCGGGATCACTCTCGGCATGGACGAGCT
GTACAAGTACTCAGATCTCGAGCTCAAGTAGACGCTTCGCTCAAATTaaaagaagagcgaattc
>CVB3_spacers_GFP
AAGCTTTAATACGACTCACTATAGGGCCGAGCGAAGCGTAAAGAAAAGAAAAAGAAAAAAGTTAAAAC
AGCCTGTGGGTTGATCCCACCCACAGGCCCATTGGGCGCTAGCACTCTGGTATCACGGTACCTTTGTG
CGCCTGTTTTATACCCCCTCCCCCAACTGTAACTTAGAAGTAACACACACCGATCAACAGTCAGCGTG
GCACACCAGCCACGTTTTGATCAAGCACTTCTGTTACCCCGGACTGAGTATCAATAGACTGCTCACGC
GGTTGAAGGAGAAAGCGTTCGTTATCCGGCCAACTACTTCGAAAAACCTAGTAACACCGTGGAAGTTG
CAGAGTGTTTCGCTCAGCACTACCCCAGTGTAGATCAGGTCGATGAGTCACCGCATTCCCCACGGGCG
ACCGTGGCGGTGGCTGCGTTGGCGGCCTGCCCATGGGGAAACCCATGGGACGCTCTAATACAGACATG
GTGCGAAGAGTCTATTGAGCTAGTTGGTAGTCCTCCGGCCCCTGAATGCGGCTAATCCTAACTGCGGA
GCACACACCCTCAAGCCAGAGGGCAGTGTGTCGTAACGGGCAACTCTGCAGCGGAACCGACTACTTTG
GGTGTCCGTGTTTCATTTTATTCCTATACTGGCTGCTTATGGTGACAATTGAGAGATCGTTACCATAT
AGCTATTGGATTGGCCATCCGGTGACTAATAGAGCTATTATATATCCCTTTGTTGGGTTTATACCACT
TAGCTTGAAAGAGGTTAAAACATTACAATTCATTGTTAAGTTGAATACAGCAAAATGGTGAGCAAGGG
CGAGGAGCTGTTCACCGGGGTGGTGCCCATCCTGGTCGAGCTGGACGGCGACGTAAACGGCCACAAGT
TCAGCGTGTCCGGCGAGGGCGAGGGCGATGCCACCTACGGCAAGCTGACCCTGAAGTTCATCTGCACC
ACCGGCAAGCTGCCCGTGCCCTGGCCCACCCTCGTGACCACCCTGACCTACGGCGTGCAGTGCTTCAG
CCGCTACCCCGACCACATGAAGCAGCACGACTTCTTCAAGTCCGCCATGCCCGAAGGCTACGTCCAGG
AGCGCACCATCTTCTTCAAGGACGACGGCAACTACAAGACCCGCGCCGAGGTGAAGTTCGAGGGCGAC
ACCCTGGTGAACCGCATCGAGCTGAAGGGCATCGACTTCAAGGAGGACGGCAACATCCTGGGGCACAA
GCTGGAGTACAACTACAACAGCCACAACGTCTATATCATGGCCGACAAGCAGAAGAACGGCATCAAGG
TGAACTTCAAGATCCGCCACAACATCGAGGACGGCAGCGTGCAGCTCGCCGACCACTACCAGCAGAAC
ACCCCCATCGGCGACGGCCCCGTGCTGCTGCCCGACAACCACTACCTGAGCACCCAGTCCGCCCTGAG
CAAAGACCCCAACGAGAAGCGCGATCACATGGTCCTGCTGGAGTTCGTGACCGCCGCCGGGATCACTC
TCGGCATGGACGAGCTGTACAAGTACTCAGATCTCGAGCTCAAGTAGAAAGAAAAGAAAAAGAAAAAA
GACGCTTCGCTCAAATTAAAAGAAGAGCGAATTC
>CVB3_minE_GFP
AAGCTTTAATACGACTCACTATAGGGCCGAGCGAAGCGTAAAGAAAAGAAAAAGAAAAAAGTTAAAAC
AGCCTGTGGGTTGATCCCACCCACAGGCCCATTGGGCGCTAGCACTCTGGTATCACGGTACCTTTGTG
CGCCTGTTTTATACCCCCTCCCCCAACTGTAACTTAGAAGTAACACACACCGATCAACAGTCAGCGTG
GCACACCAGCCACGTTTTGATCAAGCACTTCTGTTACCCCGGACTGAGTATCAATAGACTGCTCACGC
GGTTGAAGGAGAAAGCGTTCGTTATCCGGCCAACTACTTCGAAAAACCTAGTAACACCGTGGAAGTTG
CAGAGTGTTTCGCTCAGCACTACCCCAGTGTAGATCAGGTCGATGAGTCACCGCATTCCCCACGGGCG
ACCGTGGCGGTGGCTGCGTTGGCGGCCTGCCCATGGGGAAACCCATGGGACGCTCTAATACAGACATG
GTGCGAAGAGTCTATTGAGCTAGTTGGTAGTCCTCCGGCCCCTGAATGCGGCTAATCCTAACTGCGGA
GCACACACCCTCAAGCCAGAGGGCAGTGTGTCGTAACGGGCAACTCTGCAGCGGAACCGACTACTTTG
GGTGTCCGTGTTTCATTTTATTCCTATACTGGCTGCTTATGGTGACAATTGAGAGATCGTTACCATAT
AGCTATTGGATTGGCCATCCGGTGACTAATAGAGCTATTATATATCCCTTTGTTGGGTTTATACCACT
TAGCTTGAAAGAGGTTAAAACATTACAATTCATTGTTAAGTTGAATACAGCAAAATGGTATCAAAAGG
AGAAGAACTATTCACAGGAGTAGTACCAATATTAGTAGAACTAGACGGGGACGTAAACGGACATAAAT
TCTCAGTATCAGGAGAAGGAGAAGGGGACGCAACATACGGAAAACTAACATTAAAATTTATCTGCACA
ACAGGCAAACTACCAGTACCATGGCCAACGTTGGTAACAACACTAACATACGGAGTGCAATGCTTCTC
AAGATACCCAGACCACATGAAACAACACGACTTCTTCAAATCAGCAATGCCAGAAGGATACGTACAAG
AAAGAACAATATTCTTCAAAGACGACGGAAACTACAAAACAAGAGCAGAAGTAAAATTTGAAGGAGAC
ACATTAGTAAACAGAATAGAATTAAAAGGAATAGACTTCAAAGAGGACGGCAACATACTAGGACACAA
ACTAGAATACAACTACAATAGCCACAACGTATACATAATGGCAGACAAACAAAAAAATGGAATCAAAG
TAAACTTCAAAATAAGACACAACATAGAGGACGGCTCAGTACAACTAGCCGACCACTACCAACAAAAC
ACACCAATAGGGGACGGACCTGTATTACTACCCGACAACCACTATCTATCAACACAATCAGCACTATC
CAAAGACCCAAACGAAAAAAGAGATCACATGGTACTACTAGAGTTCGTAACAGCAGCAGGAATAACAT
TAGGAATGGACGAACTATACAAATACTCAGACCTAGAATTAAAATAAAAAGAAAAGAAAAAGAAAAAA
GACGCTTCGCTCAAATTAAAAGAAGAGCGAATTC

### EXAMPLES

### Example 1: Design of circRNAs

FIG. 1 shows a schematic for methods of enhancing RNA circularization via non-complementary and complementary sequence elements. Specifically, there is a 5′ and 3′ overhang upstream of a complementarity region that drives circularization, e.g. via an RNA ligase.

FIG. 1 shows predicted secondary structure from various 5′ and 3′ RNA base additions. Circled region shows 5′ most nucleotide. Three versions are shown and tested herein: loose hairpin, tight hairpin and free/bound.

### Example 2: Construction of circRNAs

circRNAs as described in FIG. 2 were synthesized and evaluated via gel electrophoresis. FIG. 2 shows that the approach of using free ends followed by bound ends (e.g. FIG. 1) enables efficient generation of circular RNA.

FIG. 3 is a gel showing the generation of circRNA (refer to FIG. 2 for conditions).

FIG. 4 shows RNA products during circle RNA synthesis. Lane 1: in vitro transcription reaction. Lane 2: Overnight ligation of ivT product using T4 RNA Ligase 1. Lane 3: Ligation product following calf intestinal phosphatase treatment. Lane 4: Ligation product following calf intestinal phosphatase treatment and RNase R digestion of the linear products.

### Example 3: Transfections with circRNA

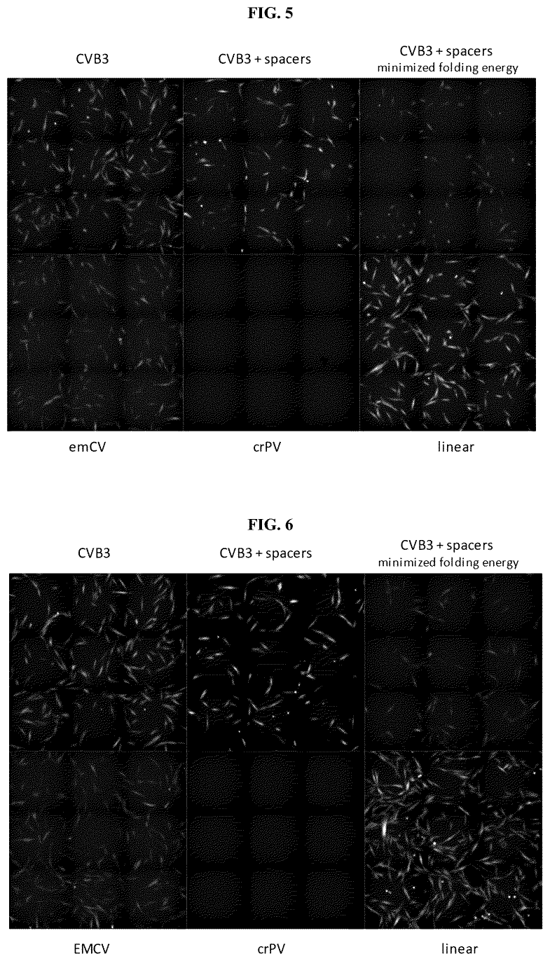

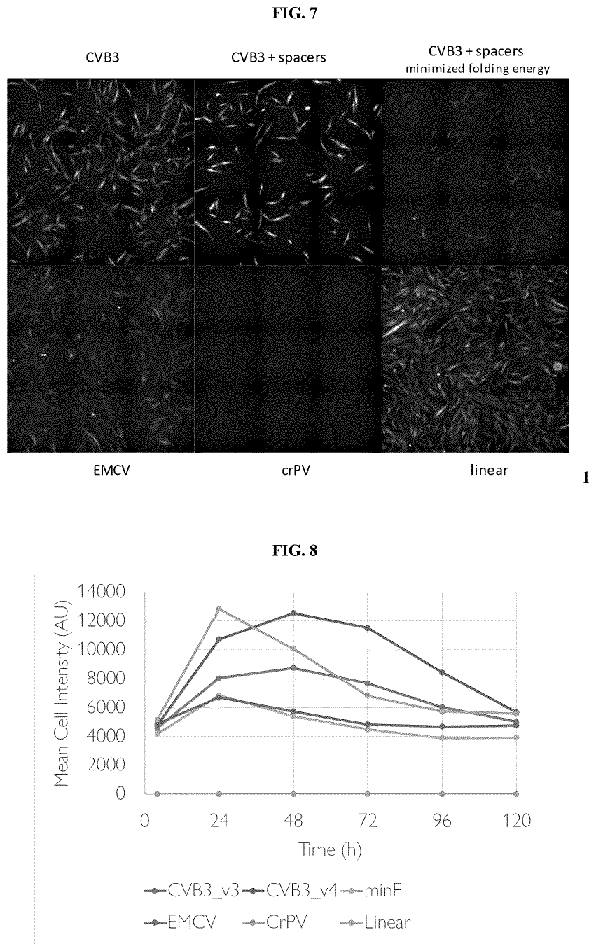

circRNA was tested for its efficiency in transfection of cells. FIG. 5 shows experiments in which 120 k of primary human dermal fibroblasts were electroporated with 750 ng of circular RNA with the indicated IRES or linear RNA encoding GFP. Representative images taken 24 h following electroporation are shown. FIG. 6 shows experiments in which 120 k of primary human dermal fibroblasts were electroporated with 750 ng of circular RNA with the indicated IRES or linear RNA encoding GFP. Representative images taken 48 h following electroporation are shown. FIG. 7 shows GFP expression 72 hours post electroporation. 120 k of primary human dermal fibroblasts were electroporated with 750 ng of circular RNA with the indicated IRES or linear RNA encoding GFP. Representative images taken 72 h following electroporation are shown.

FIG. 8 shows mean cell fluorescence intensity vs. time. For clarity, at time point 72, the curves are, top to bottom: CVB3_v4; CVB3_v3, linear, EMCV, minE, and CrPV. 120 k primary human dermal fibroblasts were electroporated with 750 ng of circular RNA with the indicated IRES or linear RNA encoding GFP. Cells were imaged at the indicated time point following electroporation and the mean intensity of each green cell was calculated.

What is claimed is:

1. A composition comprising a nucleic acid of the structure:
5′-X—Y-A-IRES-B—CDS—C—Y′—Z-3′,
wherein
Y and Y′ each independently comprise one or more nucleotides and Y and Y′ are substantially complementary;
X and Z each independently comprise one or more nucleotides and X and Z are not substantially complementary;
IRES comprises an internal ribosome entry site;
CDS comprises a coding sequence; and
A, B, and C are each independently a spacer comprising one or more nucleotides or null.

2. The composition of claim 1, wherein the nucleic acid is RNA.

3. The composition of claim 2, wherein the RNA is synthetic RNA.

4. The composition of any one of claims 1-3, wherein the nucleic acid forms a circular RNA (circRNA), e.g., when contacted with an RNA ligase.

5. The composition of any one of claims 1-4, wherein the CDS comprises one or more exons.

6. The composition of any one of claims 1-5, wherein the CDS encodes one or more proteins of interest.

7. The composition of claim 6, wherein the protein of interest is a soluble protein.

8. The composition of claim 6 or claim 7, wherein the protein of interest is selected from Table 1, Table 2, or Table 3, inclusive of the protein product of any gene of Table 1, Table 2, or Table 3.

9. The composition of claim 6 or claim 7, wherein the protein of interest is one or more reprogramming factors, optionally selected from Oct4, Sox2, Klf4, c-Myc, l-Myc, Tert, Nanog, Lin28, Glis1, Utf1, Aicda, miR200 micro-RNA, miR291 micro-RNA, miR294 micro-RNA and miR295 micro-RNA, miR302 micro-RNA, miR367 micro-RNA, miR369 micro-RNA, or a natural or engineered variant, family member, orthologue, fragment or fusion construct thereof.

10. The composition of claim 6 or claim 7, wherein the protein of interest is one or more gene-editing proteins, optionally selected from a nuclease, a transcription activator-like effector nuclease (TALEN), a zinc-finger nuclease, a meganuclease, a nickase, a clustered regularly interspaced short palindromic repeat (CRISPR)-associated protein, CRISPR/Cas9, Cas9, xCas9, Cas12a (Cpf1), Cas13a, Cas14, CasX, CasY, a Class 1 Cas protein, a Class 2 Cas protein, and MAD7, or a natural or engineered variant, family member, orthologue, fragment or fusion construct thereof.

11. The composition of claim 6 or claim 7, wherein the protein of interest is one or more gene-editing proteins comprising (i) a DNA-binding domain comprising a plurality of repeat sequences and at least one of the repeat sequences comprises the amino acid sequence: LTPvQVVAIAwxyzGHGG or LTPvQVVAIAwxyzGTHG and is between 36 and 39 amino acids long, wherein: “v” is Q, D or E, “w” is S or N, “x” is H, N, or I, “y” is D, A, I, N, G, H, K, S, or null, and “z” is GGKQALETVQRLLPVLCQD or GGKQALETVQRLLPVLCQA and (ii) a nuclease domain comprising a catalytic domain of a nuclease.

12. The composition of claim 6 or claim 7, wherein the protein of interest is one or more gene-editing proteins comprising (i) a DNA-binding domain comprising a plurality of repeat sequences and at least one of the repeat sequences comprises the amino acid sequence: LTPvQVVAIAwxyzα and is between 36 and 39 amino acids long, wherein: v is Q, D or E, w is S or N, x is I, H, N, or I, y is D, A, I, N, H, K, S, G or null, z is GGRPALE, GGKQALE, GGKQALETVQRLLPVLCQDHG, GGKQALETVQRLLPVLCQAHG, GKQALETVQRLLPVLCQDHG, GKQALETVQRLLPVLCQAHG, GGKQALETVQRLLPVLCQD or GGKQALETVQRLLPVLCQA, a is four consecutive amino acids; and (ii) a nuclease domain comprising a catalytic domain of a nuclease.

13. The composition of claim 12, wherein a is selected from GHGG, HGSG, HGGG, GGHD, GAHD, AHDG, PHDG, GPHD, GHGP, PHGG, PHGP, AHGA, LHGA, VHGA, IVHG, IHGM, RHGD, RDHG, RHGE, HRGE, RHGD, HRGD, GPYE, NHGG, THGG, GTHG, GSGS, GSGG, GGGG, GRGG, and GKGG.

14. The composition of any one of claims 1-13, wherein the IRES is selected from cMyc, CVB3 (coxsackievirus B3), EMCV (encephalomyocarditis virus), HCV (hepatitis C virus) HRV (human rhinovirus), or a natural or engineered variant, family member, orthologue, fragment or fusion construct thereof.

15. The composition of any one of claims 1-13, wherein the IRES is selected from poliovirus (PV), encephalomyelocarditis virus (EMCV), classical swine-fever virus (CSFV), foot-and-mouth disease virus (FMDV), human immunodeficiency virus (HIV), bovine viral diarrhoea virus (BVDV) and cricket paralysis virus (CrPV), or a natural or engineered variant, family member, orthologue, fragment or fusion construct thereof.

16. The composition of any one of claims 1-15, wherein the nucleic acid further comprises a Type IIS restriction enzyme site at or near the 3′ end.

17. The composition of any one of claims 1-16, wherein A comprises one or more nucleotides.

18. The composition of any one of claims 1-16, wherein the A is null.

19. The composition of any one of claims 1-18, wherein Y and Y′ are fully complementary.

20. The composition of any one of claims 1-18, wherein Y and Y′ are partially complementary.

21. The composition of any one of claims 1-20, wherein X, Z, Y and Y′ form a hairpin structure.

22. The composition of claim 21, wherein the hairpin structure is a loose hairpin.

23. The composition of claim 21, wherein the hairpin structure is a tight hairpin.

24. The composition of any one of claims 1-20, wherein X, Z, Y and Y′ do not form a hairpin structure.

25. The composition of any one of claims 1-24, wherein X, Z, Y and Y′ form a structure of FIG. 2.

26. The composition of any one of claims 1-25, wherein X and Z are suitable for interaction with an RNA ligase.

27. The composition of claim 26, wherein the RNA ligase is a single strand RNA ligase.

28. The composition of claim 27, wherein the single strand RNA ligase is T4 RNA Ligase 1, or a natural or engineered variant, family member, orthologue, fragment or fusion construct thereof.

29. The composition of any one of claims 4-28, wherein the circRNA is substantially stable from an exonuclease.

30. The composition of any one of claims 1-29, wherein the nucleic acid is suitable for synthesis by in vitro transcription.

31. A pharmaceutical composition comprising the nucleic acid composition of any one of claims 1-30 or comprising the circRNA composition of any one of claims 4-30, and a pharmaceutically acceptable carrier, vehicle or excipient.

32. A host cell comprising the composition of any one of claims 1-30.

33. A method of making a circRNA comprising contacting the nucleic acid composition of any one of claims 1-30 with one or more RNA ligases to result in circularization of the nucleic acid and formation of the circRNA.

34. The method of claim 33, wherein the RNA ligase is a single strand RNA ligase.

35. The method of claim 34, wherein the single strand RNA ligase is T4 RNA Ligase 1, or a natural or engineered variant, family member, orthologue, fragment or fusion construct thereof.

36. The method of any one of claims 33-35, wherein the nucleic acid is synthesized by in vitro transcription.

37. The method of any one of claims 33-36, wherein the nucleic acid comprises at least one non-canonical nucleotide, optionally selected from 5-methylcytidine, 5-hydroxycytidine, 5-hydroxymethylcytidine, 5-carboxycytidine, 5-formylcytidine, 5-methoxycytidine, pseudouridine, 5-hydroxyuridine, 5-methyluridine, 5-hydroxymethyluridine, 5-carboxyuridine, 5-methoxyuridine, 5-formyluridine, 5-hydroxypseudouridine, 5-methylpseudouridine, 5-hydroxymethylpseudouridine, 5-carboxypseudouridine, 5-methoxypseudouridine, and 5-formylpseudouridine.

38. The method of any one of claims 33-36, wherein the nucleic acid lacks any non-canonical nucleotides.

39. A method of expressing a protein of interest in a cell, comprising contacting the cell with the nucleic acid composition of any one of claims 1-30 or the circRNA composition of any one of claims 4-30.

40. The method of claim 39, wherein the cell is contacted with mild hypothermic conditions.

41. The method of claim 40, wherein the mild hypothermic conditions are about 30° C. to about 36° C.

42. A method of gene editing a target nucleic acid in a cell, comprising contacting the cell with the composition or pharmaceutical composition of any one of claims 10-31, wherein:
a. the composition comprises a circRNA and
b. the CDS encodes one or more proteins of interest, the proteins of interest being one or more gene-editing proteins, optionally selected from a nuclease, a transcription activator-like effector nuclease (TALEN), a zinc-finger nuclease, a meganuclease, a nickase, a clustered regularly interspaced short palindromic repeat (CRISPR)-associated protein, CRISPR/Cas9, Cas9, xCas9, Cas12a (Cpf1), Cas13a, Cas14, CasX, CasY, a Class 1 Cas protein, a Class 2 Cas protein, MAD7, and a gene-editing protein comprising a repeat sequence comprising LTPvQVVAIAwxyzα, or a natural or engineered variant, family member, orthologue, fragment or fusion construct thereof
c. wherein the gene-editing protein is directed to the target nucleic acid.

43. The method of claim 42, wherein the cell is contacted with mild hypothermic conditions, e.g., about 30° C. to about 36° C.

44. The method of claim 42 or 43, wherein the target nucleic acid is a gene selected from Table 2 or encodes a peptide or protein selected from Table 1 or Table 3.

45. The method of any one of claims 42-44, wherein the method further comprises reprogramming the cell, e.g., comprising contacting the cell with a composition of any one claim 9 or 14-30.

46. A method of reprogramming a cell, comprising contacting the cell with the composition or pharmaceutical composition of any one of claim 9 or 14-31, wherein:
a. the composition comprises a circRNA and
b. the CDS encodes one or more proteins of interest, the protein of interest being one or more reprogramming factors, optionally selected from Oct4, Sox2, Klf4, c-Myc, l-Myc, Tert, Nanog, Lin28, Glis1, Utf1, Aicda, miR200 micro-RNA, miR291 micro-RNA, miR294 micro-RNA and miR295 micro-RNA, miR302 micro-RNA, miR367 micro-RNA, miR369 micro-RNA, or a natural or engineered variant, family member, orthologue, fragment or fusion construct thereof.

47. The method of claim 46, wherein the method comprises (a) providing a differentiated or a non-pluripotent cell; (b) culturing the differentiated or a non-pluripotent cell; (c) transfecting the differentiated or a non-pluripotent cell with the circRNA.

48. The method of claim 47, wherein the transfecting is via electroporation.

49. The method of any one of claims 46-48, wherein step (c) occurs in the presence of a medium containing ingredients that support reprogramming of the differentiated or a non-pluripotent to a less differentiated state.

50. The method of any one of claims 46-49, further comprising repeating step (c) at least twice during 5 consecutive days.

51. The method of claim 50, wherein the amount of one or more circRNA molecules transfected in one or more later transfections is greater than the amount transfected in one or more earlier transfections.

52. The method of any one of claims 46-51, wherein steps (a)-(c) are performed without using feeder cells and occur in the presence of a feeder cell conditioned medium.

53. The method of any one of claims 46-51, wherein step (c) is performed without using irradiated human neonatal fibroblast feeder cells and occurs in the presence of a feeder cell conditioned medium.

54. The method of any one of claims 46-53, wherein the circRNA molecule encodes two, three, four, five, six, seven, eight, nine, ten, eleven, or more reprogramming factor(s) selected from Oct4, Sox2, Klf4, c-Myc, l-Myc, Tert, Nanog, Lin28, Glis1, Utf1, Aicda, miR200 micro-RNA, miR291 micro-RNA, miR294 micro-RNA and miR295 micro-RNA, miR302 micro-RNA, miR367 micro-RNA, miR369 micro-RNA and biologically active fragments, analogues, variants and family members thereof.

55. The method of any one of claims 47-54, wherein the differentiated or the non-pluripotent cell is derived from a biopsy.

56. The method of any one of claims 47-55, wherein the differentiated or the non-pluripotent cell is from a human subject.

57. The method of claim 55, wherein the differentiated or the non-pluripotent cell is derived from a dermal punch biopsy sample.

58. The method of any one of claims 47-57, wherein the differentiated or the non-pluripotent cell is a skin cell, e.g., a fibroblast or a keratinocyte.

59. The method of any one of claims 46-58, further comprising contacting the cell or further contacting the cell with at least one member of the group: poly-L-lysine, poly-L-ornithine, RGD peptide, fibronectin, vitronectin, collagen, and laminin.

60. The method of any one of claims 46-59, wherein the circRNA molecule comprises at least one non-canonical nucleotide, optionally selected from 5-methylcytidine, 5-hydroxycytidine, 5-hydroxymethylcytidine, 5-carboxycytidine, 5-formylcytidine, 5-methoxycytidine, pseudouridine, 5-hydroxyuridine, 5-methyluridine, 5-hydroxymethyluridine, 5-carboxyuridine, 5-methoxyuridine, 5-formyluridine, 5-hydroxypseudouridine, 5-methylpseudouridine, 5-hydroxymethylpseudouridine, 5-carboxypseudouridine, 5-methoxypseudouridine, and 5-formylpseudouridine.

61. The method of any one of claims 46-59, wherein the circRNA molecule lacks any non-canonical nucleotides.

62. The method of any one of claims 49-61, wherein the medium is substantially free of immunosuppressants.

63. The method of any one of claims 46-62, wherein the cell is contacted with mild hypothermic conditions, e.g., about 30° C. to about 36° C.

64. The method of any one of claims 49-63, wherein the method further comprises gene-editing the cell, e.g., comprising contacting the cell with a composition of any one claims 10-30.

65. The method of claim 64, wherein the gene-editing targets a nucleic acid which is a gene selected from Table 2 or which encodes a peptide or protein selected from Table 1 or Table 3.

66. A method of treating a disease, disorder, or condition comprising:
(a) contacting a cell with the composition or pharmaceutical composition of any one of claims 1-31 and administering the cell to a patient in need thereof, or
(b) administering the composition or pharmaceutical composition of any one of claims 1-31 to a patient in need thereof.

67. The method of claim 66, wherein the disease, disorder, or condition is selected from Table 1 or Table 3.

68. The method of any one of claims 33 to 67, wherein the circRNA molecule encodes one or more of: a T-cell receptor, a chimeric antigen receptor, a bispecific T-cell engagers (BiTE), a checkpoint inhibitor, an antibody, a nanobody, or a natural or engineered variant, family member, orthologue, fragment or fusion construct thereof.

69. The method of any one of claims 33 to 67, wherein the circRNA molecule encodes one or more of: Interleukin 7, Interleukin 12, Interleukin 15, Interleukin 18, dominant-negative TGF-β receptor II (dnTGF-βRII), constitutively active Akt (caAkt), or CD40 Ligand (CD40L), or a natural or engineered variant, family member, orthologue, fragment or fusion construct thereof.

70. The method of any one of claims 33 to 67, wherein the circRNA encodes a protein of interest that serves as a vaccine when introduced into a subject in need of vaccination.

71. The method of any one of claims 42 to 45 or claims 64 to 67, wherein the gene editing further comprises transfecting the cell with a nucleic acid that acts as a repair template.

72. The method of claim 71, wherein the repair template either causes insertion of a DNA sequence in the region of a gene edit, e.g., a single-strand or double-strand break, or causes the DNA sequence in the region of the gene edit to otherwise change.

73. The method of any one of claims 42 to 45, 64 to 67, 71, or 72, wherein the gene edit targets a genomic safe harbor locus, e.g., TRAC and AAVS1.

74. The method of any one of claims 46 to 67, wherein the reprogrammed cell is provided to a subject in need thereof in the context of a stem cell therapy.
